SEQUENCE LISTING (33 sequences)

SEQ ID NO: 1 - RNA, 20 nt, synthetic construct
  source 1..20: mol_type = other RNA
cgtagcccacggcgatgaag

SEQ ID NO: 2 - RNA, 20 nt, synthetic construct
  source 1..20: mol_type = other RNA
atttcttcacatccgtgtcc

SEQ ID NO: 3 - RNA, 20 nt, synthetic construct
  source 1..20: mol_type = other RNA
gtctcctggtcccaatactc

SEQ ID NO: 4 - RNA, 20 nt, synthetic construct
  source 1..20: mol_type = other RNA
gtagcccacggcgatgaagc

SEQ ID NO: 5 - RNA, 20 nt, synthetic construct
  source 1..20: mol_type = other RNA
tagcccacggcgatgaagcg

SEQ ID NO: 6 - RNA, 20 nt, synthetic construct
  source 1..20: mol_type = other RNA
gagggttcggggcgccatga

SEQ ID NO: 7 - RNA, 20 nt, synthetic construct
  source 1..20: mol_type = other RNA
ccggaacacacggaatgtga

SEQ ID NO: 8 - RNA, 20 nt, synthetic construct
  source 1..20: mol_type = other RNA
acagcgacgccgcgagccag

SEQ ID NO: 9 - RNA, 20 nt, synthetic construct
  source 1..20: mol_type = other RNA
ccactcacagattgaccgag

SEQ ID NO: 10 - RNA, 20 nt, synthetic construct
  source 1..20: mol_type = other RNA
cgacgccgcgagccagagga

SEQ ID NO: 11 - RNA, 20 nt, synthetic construct
  source 1..20: mol_type = other RNA
cagattgaccgagtggacct

SEQ ID NO: 12 - RNA, 20 nt, synthetic construct
  source 1..20: mol_type = other RNA
cagactgaccgagtggacct

SEQ ID NO: 13 - RNA, 20 nt, synthetic construct
  source 1..20: mol_type = other RNA
acagactgaccgagtggacc

SEQ ID NO: 14 - RNA, 20 nt, synthetic construct
  source 1..20: mol_type = other RNA
gccgggccgggacacggatg

SEQ ID NO: 15 - RNA, 20 nt, synthetic construct
  source 1..20: mol_type = other RNA
ccaggagacacggaatgtga

SEQ ID NO: 16 - RNA, 20 nt, synthetic construct
  source 1..20: mol_type = other RNA
ccagtcacagactgaccgag

SEQ ID NO: 17 - RNA, 20 nt, synthetic construct
  source 1..20: mol_type = other RNA
tcgacagcgacgccgcgagc

SEQ ID NO: 18 - RNA, 20 nt, synthetic construct
  source 1..20: mol_type = other RNA
gagatacatctacaaccggg

SEQ ID NO: 19 - RNA, 20 nt, synthetic construct
  source 1..20: mol_type = other RNA
tggagagatacatctacaac

SEQ ID NO: 20 - RNA, 20 nt, synthetic construct
  source 1..20: mol_type = other RNA
ggttgtagatgtatctctcc

SEQ ID NO: 21 - RNA, 20 nt, synthetic construct
  source 1..20: mol_type = other RNA
ggagagatacatctacaacc

SEQ ID NO: 22 - RNA, 20 nt, synthetic construct
  source 1..20: mol_type = other RNA
aggaatgctacgcgtttaat

SEQ ID NO: 23 - RNA, 20 nt, synthetic construct
  source 1..20: mol_type = other RNA
cagatacatctataaccgag

SEQ ID NO: 24 - RNA, 20 nt, synthetic construct
  source 1..20: mol_type = other RNA
cgagtactggaacagccaga

SEQ ID NO: 25 - RNA, 20 nt, synthetic construct
  source 1..20: mol_type = other RNA
cccgttggtgaagtagcaca

SEQ ID NO: 26 - RNA, 20 nt, synthetic construct
  source 1..20: mol_type = other RNA
gtgctacttcaccaacggga

SEQ ID NO: 27 - RNA, 20 nt, synthetic construct
  source 1..20: mol_type = other RNA
aggtcgtgcggagctccaac

SEQ ID NO: 28 - RNA, 20 nt, synthetic construct
  source 1..20: mol_type = other RNA
aggtgacagttgctacccga

SEQ ID NO: 29 - RNA, 20 nt, synthetic construct
  source 1..20: mol_type = other RNA
tcccgttggtgaagtagcac

SEQ ID NO: 30 - RNA, 20 nt, synthetic construct
  source 1..20: mol_type = other RNA
aactacgaggtggcgtaccg

SEQ ID NO: 31 - RNA, 20 nt, synthetic construct
  source 1..20: mol_type = other RNA
ctgttccagtactcggcaac

SEQ ID NO: 32 - RNA, 20 nt, synthetic construct
  source 1..20: mol_type = other RNA
actacgaggtggcgtaccgc

SEQ ID NO: 33 - RNA, 20 nt, synthetic construct
  source 1..20: mol_type = other RNA
cgaagcgcacgtactcctct

(19) United States
(12) Patent Application Publication
(10) Pub. No.: US 2025/0368955 A1
(43) Pub. Date: Dec. 4, 2025

(54) B CELL LINEAGES DERIVED FROM PLURIPOTENT CELLS

(71) Applicant: Stratus Therapeutics, Inc., Cambridge, MA (US)

(72) Inventor: Dhvanit Shah, Chestnut Hill, MA (US)

(21) Appl. No.: 19/117,872

(22) PCT Filed: Oct. 5, 2023
(86) PCT No.: PCT/US2023/076111

§ 371 Date: Apr. 2, 2025

Related U.S. Application Data
(60) Provisional application No. 63/413,454, filed on Oct. 5, 2022.

(51) Int. Cl. C12N 5/0781; A61K 35/17; A61K 40/13; A61K 40/31; A61P 35/00; C07K 14/74; C12N 5/10

(52) U.S. Cl. CPC C12N 5/0635; A61K 35/17; A61K 40/13; A61K 40/31; A61P 35/00; C07K 14/70539; C12N 5/10; C12N 2506/11; C12N 2506/45; C12N 2510/00

(57) ABSTRACT

The present disclosure provides for efficient ex vivo processes for generating B cell lineages from human induced pluripotent stem cells (iPSCs). Cells generated according to the disclosure in various embodiments are functional and/or more closely resemble the corresponding lineage isolated from peripheral blood or lymphoid organs. The present invention in some aspects provides isolated cells and cell compositions produced by the methods disclosed herein, as well as methods for cell therapy.

### CROSS-REFERENCE TO RELATED APPLICATIONS

This application claims the benefit of and priority to U.S. Provisional Patent Application No. 63/413,454 filed Oct. 5, 2022, the contents of which are hereby incorporated by reference in their entirety.

### SEQUENCE LISTING

The instant application contains a Sequence Listing which has been submitted in XML format via EFS-Web and is hereby incorporated by reference in its entirety. Said XML copy, created on Sep. 26, 2023, is named GRU-017PC_Sequence_Listing.xml and is 30,036 bytes in size.

### BACKGROUND

B cell lineages play a crucial role in tissue maintenance and help orchestrate effector and regulator immune responses. However, their clinical use as a cell therapy is hampered by the small numbers of such cells that can be isolated from regular leukapheresis products. Therefore, development of large scale, off-the-shelf, B lymphocyte lineages would be an attractive tool to fight cancer and infectious diseases, among others.

### SUMMARY OF THE DISCLOSURE

The present disclosure, in various aspects and embodiments, provides methods for generating hematopoietic lineages for cell therapy, including B cells or progenitors thereof, as well as their precursors. In various embodiments, the invention provides for efficient ex vivo processes for developing such hematopoietic lineages from human induced pluripotent stem cells (iPSCs), including gene edited iPSCs. Cells generated according to the disclosure in various embodiments are functional and/or more closely resemble the corresponding lineage isolated from peripheral blood or bone marrow. The present invention also provides isolated cells and cell compositions produced by the methods disclosed herein, as well as methods for cell therapy.

In one aspect, the disclosure provides a method for preparing a cell population comprising myeloid cells of the innate immune system. The method comprises preparing a pluripotent stem cell (PSC) population, such as an induced pluripotent stem cell (iPSC) population differentiated to embryoid bodies, and enriching for CD34+ cells to thereby prepare a CD34+-enriched population. Endothelial-to-hematopoietic transition (EHT) is induced in the CD34+-enriched population to thereby prepare a hematopoietic stem cell (HSC) population, optionally followed by a further enrichment of CD34+ cells. The resulting HSC population (or fraction thereof) can be differentiated to a myeloid lineage of the innate immune system (e.g., phagocytic cells or their precursors). In some embodiments, the disclosure provides a method for generating B cells, B-CAR cells, and immature and mature B cells (or their precursors) from the HSC population ex vivo.

In various embodiments, the iPSCs are prepared by reprogramming somatic cells. In some embodiments, iPSCs are generated from somatic cells such as (but not limited to) fibroblasts or PBMCs (or cells isolated therefrom). In some embodiments, iPSCs are derived from CD34+ cells isolated from peripheral blood.

In various embodiments, the iPSCs are gene edited to assist in HLA matching, such as deletion of one or more HLA Class I and/or Class II alleles. For example, iPSCs can be gene edited to delete one or more of HLA-A, HLA-B, and HLA-C, and to delete one or more of HLA-DP, HLA-DQ, and HLA-DR. In certain embodiments, the iPSCs retain expression of at least one HLA Class I and at least one HLA Class II complex. In certain embodiments, iPSCs are homozygous for at least one retained Class I and Class II loci. In some embodiments, the iPSCs are gene edited to be HLA-A^{neg}, homozygous for both HLA-B and HLA-C, and ILA-DPB1^{neg} and HLA-DQB1^{neg}. In some embodiments, the iPSCs are further homozygous for HLA-DRB1.

In various embodiments, iPSCs are prepared, and expanded using a culture system. Expanded iPSCs can be recovered from the culture for generating embryoid bodies (EBs). EBs, created by differentiation of the iPSCs, are three-dimensional aggregates of iPSCs and comprise the three (or alternatively two or one) embryonic germ layer(s) based on the differentiation method(s). In some embodiments, the process comprises harvesting CD34+-enriched cells from the EBs and inducing endothelial-to-hematopoietic differentiation.

In some embodiments, iPSC differentiation proceeds until cells are at least about 20% CD34+ or at least about 30% CD34+. In some embodiments, CD34 enrichment and EHT may be induced at Day 7 to Day 14 of iPSC differentiation. Differentiation of iPSCs can be according to known techniques. In some embodiments, iPSC differentiation involves factors such as, but not limited to, combinations of Y-27632, TPO, IL-3, SCF, IL-6, IL-11, IGF-1, VEGF, bFGF, BMP4, and FLT3.

Induction of EHT can be with any known process. In some embodiments, induction of EHT generates a hematopoietic stem cell (HSC) population comprising LT-HSCs. In some embodiments, EHT generates HSCs through endothelial or hemogenic endothelial cell (HEC) precursors using mechanical, biochemical, pharmacological and/or genetic means (e.g., via stimulation, inhibition, and/or genetic modifications). In some embodiments, the EHT generates a stem cell population comprising one or more of long-term hematopoietic stem cells (LT-HSCs), short-term hematopoietic stem cells (ST-HSCs), and hematopoietic stem progenitor cells.

In some embodiments, the method comprises increasing the expression or activity of DNA (cytosine-5-)-methyltransferase 3 beta (Dnmt3b) in PSCs, embryoid bodies, CD34+-enriched cells, ECs, HECs or HSCs, which can be by mechanical, genetic, biochemical, or pharmacological means. For example, in some embodiments, cells are contacted with an effective amount of an agonist of a mechanosensitive receptor or a mechanosensitive channel that increases the activity or expression of Dnmt3b. In some embodiments, the mechanosensitive receptor is Piezol. An exemplary Piezol agonist is Yoda1. In various embodiments, pharmacological Piezol activation is applied to CD34+ cells harvested from EBs. In some embodiments, the process does not involve increasing the expression of dnmt3b, such as by using a Piezol agonist.

In various embodiments, CD34+ cells (e.g., the floater and/or adherent cells) are harvested from the culture undergoing endothelial-to-hematopoietic transition between Day 10 to Day 20 of iPSC differentiation, such as from Day 12 to Day 17 of iPSC differentiation. Hematopoietic stem cells (HSCs) which can give rise to innate myeloid, erythroid, and lymphoid lineages, can be identified based on the expression of CD34 and the absence of lineage specific markers (termed Lin-).

In various embodiments, the HSC population or fraction thereof is differentiated to a hematopoietic lineage, which can be selected from particularly B cell lineages and their progenitors and progenies, including multipotent progenitor cells (MIPPs), common lymphoid precursor (CLP), common lymphoid 2 progenitor (LCA-2), and B cells. The B cells produced can be early pro-B cells, late pro B cells, pre B cells, and immature B cells with the ability to generate B cells. In various embodiments, the disclosure provides for methods for ex vivo production of cell populations corresponding to transitional B cells, regulatory B cells, marginal zone B cells, follicular B cells, activated B cells, memory B cells, and plasma B cells (collectively referred to as “B cells”).

In some embodiments, the B cells are further modified to express a chimeric antigen receptor (CAR). Additionally, or optionally, the B-CAR may be engineered to express and/or secrete cytokines (e.g., IL-4, IL-6, IL-15 etc. or an interferon) to make the CAR expressing cells more potent in targeting tumors (for example). In a non-limiting example, cells can be efficiently transduced by a vector, such as but not limited to retroviral or nonintegrating viral vectors, or nonviral vectors, carrying a CAR. The CAR may target a tumor-associated antigen or marker in some embodiments.

In other aspects, the invention provides a cell population, or pharmaceutically acceptable composition thereof, comprising a B cell lineage or precursor thereof, and which may be produced by the methods described herein. In some embodiments, the cell population is capable of engraftment in a thymus, spleen, or secondary lymphoid organ upon administration to a subject in need. In various embodiments, the composition for cellular therapy is prepared that comprises the cell population and a pharmaceutically acceptable vehicle. In some embodiments, the cell population is HLA-A^{neg}, homozygous for both HLA-B and HLA-C, and HLA-DPB1^{neg} and HLA-DQB1^{neg}. In some embodiments, the cell population is further homozygous for HLA-DRB1. In various embodiments, the composition comprises myeloid lineages selected from one or more of monocytes, macrophages, dendritic cells, neutrophils, and myeloid progenitors.

In other aspects, the invention provides a method for cell therapy, comprising administering the cell population described herein, or pharmaceutically acceptable composition thereof, to a human subject in need thereof. In various embodiments, the methods described herein are used to treat blood (malignant and non-malignant), bone marrow, immune diseases, and infectious diseases. In various embodiments, the human subject has a condition comprising one or more of lymphopenia, a cancer, an immune deficiency, and an autoimmune disease.

Other aspects and embodiments of this disclosure will be apparent from the following detailed disclosure and working examples.

### BRIEF DESCRIPTION OF DRAWINGS

FIG. 1 shows that ETV2 over-expression (OE) does not affect pluripotency. FIG. 1 shows FACS plots representative of transduction efficiency of iPSC with an adenoviral vector to overexpress ETV2 and GFP sequences. ETV2 overexpression does not affect the iPSC stemness as shown by the expression of the TRA-1-60 stemness marker.

FIG. 2 shows that ETV2 over-expression (OE) increases the yield of hemogenic endothelial cells. Representative flow cytometric analysis of hemogenic endothelial cells (described as CD235a-CD34+CD31+) and relative quantification demonstrates that ETV2-OE enhances the formation of hemogenic endothelial cells.

FIG. 3 shows that ETV2 over-expression (OE) enhances CD34+ cell formation during iPSC differentiation. Representative flow cytometric analysis of CD34+ cells and relative quantification demonstrates that ETV2-OE enhances the CD34+ cell formation.

FIG. 4A and FIG. 4B show that iPSC-derived HSCs that are derived with EHT of CD34+ cells (with Piezol activation) undergo pro-T cell differentiation similar to bone marrow (BM)-HSCs. FIG. 4A is a FACS plot of differentiation efficiency to CD34+CD7+ pro T cells of Bone Marrow (BM) HSCs and iPSC-HSCs derived with EHT of CD34+ cells (with Piezol activation). FIG. 4B is a quantification of CD34+CD7+ cells (%) derived with (1) BM-HSCs and (2) iPSC-HSCs (EHT of CD34+ cells with Piezol Activation). FIG. 4B shows the average of three experiments.

FIG. 5A and FIG. 5B show that iPSC-derived HSCs generated with EHT of CD34+ cells (in this example with Piezol activation) undergo T cell differentiation and can be activated with CD3/CD28 beads similar to BM-HSCs. FIG. 5A is a FACS plot of activation efficiency (CD3+CD69+ expression) of T cells differentiated from BM-HSCs and iPSC-derived HSCs (generated with EHT of CD34+ cells with Piezol activation). FIG. 5B is a quantification of CD3+CD69+ cells (%) derived with (1) BM-HSCs and (2) iPSC-HSCs (derived with EHT of D8 CD34+ cells, with Piezol Activation). FIG. 5B shows the average of three experiments.

FIG. 6 shows that iPSC-derived HSCs generated with EHT of CD34+ cells (in this example with Piezol activation) can differentiate to functional T cells. IFNγ expression is a consequence of T cell activation after T cell receptor (TCR) stimulation via CD3/CD28 beads. IFNγ expression in T cells differentiated from iPSC-derived HSCs (with EHT of CD34+ cells, in this case generated with Piezol activation), enhances the ability to further differentiate to functional lymphocytes. FIG. 6 shows the average of three experiments.

FIG. 7A and FIG. 7B show the phenotype analysis of HLA edited (e.g., triple knockout cells performed by FACS and immunofluorescence. FIG. 7A shows the overall expression of HLA class-I molecules (HLA-A, HLA-B, and HLA-C) on the cell surface, where the HLA edited cells are positive for overall HLA class-I expression to a similar degree as wild-type cells. FIG. 7B shows cell expression of HLA-A via immunofluorescence, where HLA-A is not expressed in the HLA edited clone.

FIG. 8 shows that the HLA edited clones preserve their pluripotency (maintain trilineage differentiation), as illustrated by immunofluorescence, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

FIG. 9 shows the immune compatibility of the HLA edited HSCs. HLA edited HSCs and control HSCs (WT, B2M KO, and HLA Class II null) were co-cultured with peripheral blood mononuclear cells (PBMCs) matching HLA-B and HLA-C, but with mismatched HLA-A, and PBMC-mediated cytotoxicity was measured by an annexin V staining assay.

FIG. 10 shows in vivo engrafting potential of HLA edited HSCs. Equal proportions of mCherry HLA edited HSCs and unedited HSCs were mixed for a competitive transplant into mice, where bone marrow (BM) and peripheral blood samples were evaluated by FACS to compare the relative amounts of each cell type present in the samples.

FIGS. 11A and 11B show that deletion of HLA-A does not impact Class I peptide presentation. FIG. 11A shows a schematic representation of immunopeptidome analysis. FIG. 11B shows results of the immunopeptidome analysis, which reveals that little difference exists in the numbers of peptides and representative proteins presented by class I molecules of WT and HLA-edited cells.

FIGS. 12A and 12B show that deletion of HLA-DP and DQ does not impact Class II peptide presentation. FIG. 12A shows immunopeptidome analysis scheme. FIG. 12B shows that despite the deletion of HLA-DP and DQ, the cells preserve their ability to present a broad spectrum of peptide through HLA Class II.

FIG. 13 is a schematic representation of in vivo testing of antigen-mediated immune response: Delayed Type Hypersensitivity Assay (DTH), sensitizing stage and elimination stage respectively.

FIGS. 14A and 14B show that HLA-edited HSCs reconstitute a functional immune system as demonstrated by DTH reaction in immune deficient mice. FIG. 14A shows a delayed-type hypersensitivity assay on transplanted mice were performed, which is an assay that involves the cross-talk of different types of immune cells. Mice were sensitized by subcutaneous injection of sheep Red blood cells (antigen). A functional immune system results in the swelling of the left paw that was measured with a micro caliper. As can be seen in FIG. 14A, the non-transplant mice did not show any left paw swelling as they are immunodeficient. Conversely, the mice transplanted with Cord Blood CD34+ cells show tissue swelling and doubled the diameter of their left paw. FIG. 14B is a graphical evaluation of the results shown in FIG. 14A.

FIG. 15 shows the HSC differentiation potential into T cell subtypes. After a 35-day differentiation period pro-T cells were evaluated by cell sorting for the presence of CD4+, CD8+, and AB+ T cell populations. FIG. 15 compares the differentiation potential of bone marrow-derived CD34+ cells, embryoid body CD34+ cells, and HSCs prepared according to the present disclosure (e.g., using Piezol activation) (“gHSCs”).

FIG. 16 shows the degree of T-cell mediated cytotoxicity measured from a co-culture of HSC-derived T cells with CD19+ lymphoma cells in the presence of an anti-CD3/CD-19 bispecific antibody. T cells prepared from HSCs according to the present disclosure (“gHSC”) demonstrate a high level of cytotoxicity against the target cells.

FIG. 17 shows the ability of the HSCs to develop into pro-T cells as measured by their CD34-CD7+ markers.

FIGS. 18A and 18B demonstrates increased expression of T cell-specific transcription factors and Thymus engrafting molecules with the pro-T cells derived from HSCs according to the instant disclosure. FIG. 18A shows TCF7 mRNA expression and FIG. 18B shows CCR7 mRNA expression.

FIGS. 19A and 19B shows that HSC-derived Pro-T Cells engraft and differentiate in thymus. FIG. 19A illustrates the engraftment and analysis procedure. FIG. 19B shows FACS analysis of CD3 cell population of cells gated on CD45+ cell population, which shows the superior engraftment and differentiation potential of the HSC-derived Pro-T Cells in the thymus.

FIG. 20 shows HSC-derived T cells can be activated in vitro. Top panel shows FACS analysis of activated T cells from different sources, including from HSCs prepared according to the present disclosure. T cells of the present disclosure demonstrate comparable or superior activation as measured by increased CD107 expression. The lower panel shows Dynabeads activation, where activated T cells express inflammatory cytokines. HSC-derived T cells express higher levels of inflammatory cytokines as exemplified by TNF-alpha and interferon gamma expression levels.

FIGS. 21A and 21B show that WT and HLA-edited HSCs can differentiate to the monocyte/macrophage lineage, which also preserves the overall expression of both class I and class II molecules as identified by CD11b+CD14+ markers (FIG. 21A). FIG. 21B shows analysis of HLA-I and HLA-II on cells gated on CD11b+CD14+.

FIGS. 22A to 22C show that HLA-DQB1 and HLA-DPB1 deletion does not affect the expression of other HLA Class II molecules. FIG. 22A is a schematic showing differentiation of HLA-edited iPSCs to macrophages. FIG. 22B is an immunofluorescence experiment confirming the specific deletion of the DPB1 and DQB1 molecules. FIG. 22C shows that the same cells preserve the class II DRB1 expression.

FIG. 23 shows that HLA-edited HSCs can differentiate into megakaryocytes (MK) which can further differentiate into platelets. Images at the left show increased proportion of platelets in HSCs by light microscopy at 1000× magnification. The graph at the right shows a statistically significant increase in the proportion of platelets differentiated from HLA-edited HSCs compared to BM CD34+ and iPSC-34+ cell populations.

FIG. 24 shows the capability of HSCs to effectively differentiation into NK cells as evidenced by fluorescence-activated cell sorting (FACS) experiments, gated based on CD56 expression.

FIGS. 25A-25C show HSC-derived NK cells effectively kill tumor cells. FIG. 25A shows an experimental schematic where the HSC-derived NK cells are co-cultured with K562 HLA-null cells, and the degree of NK cell degranulation is measured using AnnexinV staining and a cytotoxicity assay. FIG. 25B shows the results of NK cell degranulation as measured by fluorescence-activated cell sorting (FACS) using AnnexinV staining. FIG. 25C shows the results of the tumor cell cytotoxicity assay, with lactate dehydrogenase (LDH) as a measure for cell death.

The term “gHSC” is used herein to refer to the iPSC-derived hematopoietic stem cells of the present disclosure.

The terms “wild type” (WT), “unedited”, “non-HLA-edited” are used interchangeability herein to refer to the non-gene edited cells of the present disclosure.

EB34+ cells refer to Embryonic body derived CD34+ cells. These comprise hemogenic endothelial cells.

### DESCRIPTION OF THE INVENTION

The present disclosure, in various aspects and embodiments, provides methods for generating hematopoietic lineages ex vivo for cell therapy, and particularly B cell lineages and their progenitors and progenies, including in various embodiments multipotent progenitor cells (MPPs), common lymphoid precursor (CLP), common lymphoid 2 progenitor (LCA-2), and B cells. B cells produced can be early pro-B cells, late pro B cells, pre B cells, and immature B cells with the ability to generate B cells. In various embodiments, the disclosure provides for methods for ex vivo production of cell populations corresponding to transitional B cells, regulatory B cells, marginal zone B cells, follicular B cells, activated B cells, memory B cells, and plasma B cells (collectively referred to as “B cells”). In various embodiments, the invention provides for efficient ex vivo processes for developing such hematopoietic lineages from human induced pluripotent stem cells (iPSCs). Cells generated according to the disclosure in various embodiments are functional and/or more closely resemble the corresponding lineage isolated from peripheral blood or lymphoid organs. The present invention also provides isolated cells and cell compositions produced by the methods disclosed herein, as well as methods for cell therapy.

In accordance with aspects and embodiments of this disclosure, the ability of human induced pluripotent stem cells (hiPSCs) to produce essentially limitless pluripotent stem cells (PSCs) is leveraged to generate boundless supply of B cell lineages or modified versions thereof (e.g., genetically modified B-CAR cells). Use of B cells as therapeutic lymphocytes have been limited by their restricted availability, cell numbers, limited expansion potential, and histocompatibility issues. Moreover, compared to primary cells, hiPSCs can more readily undergo genetic modifications in vitro, thereby offering opportunities to improve cell-target specificity, cell numbers, as well as bypassing HLA-matching issues for example. Additionally, fully engineered hiPSC clones, as compared to primary cells, can serve as a stable and safe source (Nianias and Themeli, 2019). Further, because hiPSCs, unlike human Embryonic Stem Cells (hESCs), are of non-embryonic origin, they are also free of ethical concerns and are consistent in quality. Accordingly, use of hiPSCs according to this disclosure confers several advantages over primary cells to generate therapeutic hematopoietic lineages such as B cell lineages.

In one aspect, the disclosure provides a method for preparing a cell population (e.g., ex vivo) of a B cell lineage. The method comprises preparing a pluripotent stem cell (PSC) population, such as an induced pluripotent stem cell (iPSC) population differentiated to embryoid bodies, and enriching for CD34+ cells to thereby prepare a CD34+-enriched population. Endothelial-to-hematopoietic transition (EHT) is induced in the CD34+-enriched population to thereby prepare a hematopoietic stem cell (HSC) population, optionally followed by a further enrichment of CD34+ cells. The resulting HSC population (or fraction thereof) can be differentiated to a B cell lineage.

In various embodiments, the B cell population comprises transitional B cells, regulatory B cells, marginal zone B cells, follicular B cells, activated B cells, memory B cells or plasma B cells, or a derivative thereof.

In some embodiments, the cell population comprises Transitional B cells (TrB cells) which are immature B cells, and which are precursors of mature B cells. TrB cells account for approximately 4% of all CD19+B lymphocytes in healthy individuals. They are present in peripheral blood, cord blood bone marrow, and secondary lymphoid tissues such as, lymph nodes, spleen, tonsils, and gut-associated lymphoid tissue (GALT). Human TrB cells are often characterized by a CD24^{hi}CD38^{hi} phenotype. TrB cells can be separated into subsets based on based on CD27, IgM, IgD, CD10, CD21, and CD32 expression. T1-T3 B cell subsets express low levels of CD27, whereas CD27+ TrB cells express CD27, CD24, and CD38 at high levels. In T1 B cells, the expression of IgM, CD10, and CD32 is high while that of IgD and CD21 is low. T2 B cells show moderate IgM, IgD, CD10, and CD32 expression and low CD21 expression. T3 B cells express IgM, IgD, CD10, CD21, and CD32 at low levels.

TrB cells can suppress autoreactive CD4+ T cell proliferation; suppress the production of pro-inflammatory cytokines by limiting the expansion of CD4+Th1 cells (IFN-γ and TNF-α production) and CD4+Th17 cells (IL-17 production); prevent the CD4+ T cells from differentiating into Th1 and Th17 cells and promote the conversion of effector CD4+ T cells into CD4+FoxP3+ Tregs while limiting the production of excessive pro-inflammatory cytokines. TrB cells also inhibit CD8+ T cell responses and maintain invariant nature killer T (iNKT) cells. In addition to producing anti-inflammatory factors, TrB cells can also secrete pro-inflammatory cytokines such as IL-6 and TNF-α. In some aspects and embodiments, TrB cells are closely related to IL-10-producing regulatory B cells (Bregs) in terms of phenotypical and functional similarities. TrB cells can also produce IL-10 and regulate CD4+ T cell proliferation and differentiation toward T helper (Th) effector cells.

In some embodiments, the cell population comprises Regulatory B (Breg) cells. Bregs are immunosuppressive cells that support immunological tolerance. Breg cells have also been implicated with the inhibition of excessive inflammation. Through the production of IL-10, TGF-β, and IL-35, Breg cells can suppress the differentiation of pro-inflammatory lymphocytes, such as tumor necrosis factor α (TNF-α)-producing monocytes, IL-12-producing dendritic cells, Th17 cells, Th1 cells, and cytotoxic CD8+ T cells. Breg cells can also induce the differentiation of immunosuppressive T cells, Foxp3+ T cells, and T regulatory 1 (Tr1) cells. Breg cells also support the maintenance of iNKT cells. Common markers for human Breg cells include CD19+CD24^{hi}CD38^{hi}CD1d^{hi}, CD19+CD24^{hi}CD27+, CD24^{hi}CD27+, CD19+CD24^{hi}CD27^{int}, CD19+CD24^{hi}CD38^{hi} and CD19+CD25^{hi}CD71^{hi}.

In various embodiments, the B cells have a phenotype consistent with Marginal zone B (MZB) cells. MZB cells provide a first line of defense in response to infections by blood-borne viruses and encapsulated bacteria where they rapidly produce IgM and class-switched IgG antibodies MZB cells may also produce IgM and class-switched IgG and IgA antibodies in response to commensal antigens. MZB cells mediate T cell-dependent antibody production. For example, MZB cells can mount a T cell-dependent response against microbial protein antigens. In some embodiments, the MZB-like cells derived from the iPSC are CD27+IgM+IgD+ cells or they express high levels of IgM, CD21, CD1, and CD9, and are low to negative for IgD, CD23, CD5, and CD11b or are CD27⁻CD45RB⁺ (which define MZ precursor cells).

In some embodiments, the B cells have a phenotype consistent with Follicular B cells. Follicular B cells participate in T-cell-dependent antibody responses. Additionally, follicular B cells respond to blood-borne pathogens in a T cell-independent manner. Following activation, follicular B cells differentiate into short-lived plasma cells in the periphery or enter into T cell-dependent germinal center reactions. Follicular B cells express high levels of IgD, and CD23; lower levels of CD21 and IgM; and no CD1 or CD5. Other cell surface markers that identify follicular B cells, include but are not limited to CD10, CD19, CD20, CD22, CD23, CD38^{LOW}, CXCR5+ and IgD^{high}.

B cells express B cell receptor (BCR), which upon binding to either soluble or membrane bound antigen activates the B cells. Activated BCR form microclusters and trigger downstream signaling cascades. The microcluster eventually undergoes a contraction phase and forms an immunological synapse, this allows for a stable interaction between B and T cells to provide bidirectional activation signals. Upon encountering an antigen, mature activated B cells proliferate and becomes a blasting B cell. These B cells form germinal centers. The germinal center B cells undergo somatic hypermutation and class switch recombination. Plasma cells and memory B cells with a high-affinity for the original antigen stimuli are produced. These cells are long lived and plasma cells may secrete antibody for weeks after the initial infection. One of the main transcriptional activators related to B cell activation is nuclear factor (NF)-KB. Some of the Common markers identifying activated B cells are CD19, CD25, CD30.

B cells generated according to the current disclosure can differentiate into plasma cells (ex vivo or in vivo). Plasma cells are specialized terminally differentiated B cells that synthesize and secrete antibodies to maintain humoral immunity. Plasma B cells, when it encounters a unique antigen, takes in the antigen through receptor-mediated endocytosis. Antigenic particles are transferred to the cell surface, loaded onto MHC II molecules and presented to a helper T cell. The binding of the helper T cell to the MHC II-antigen complex activates the B cell. The activated B cell goes through a period of rapid proliferation and somatic hypermutation. Selection occurs for those cells that produce antibodies with a high affinity for that particular antigen. Once terminally differentiated, the plasma B cell only secretes antibodies specific for that antigen and can no longer generate antibodies to other antigens.

B cells generated according to the current disclosure can differentiate into Memory Bells (ex vivo or in vivo). Memory B cells are B lymphocytes that remember a specific antigen, upon initial B cell response. Memory B cells are held in reserve, in the germinal centers of the lymphatic system, for when the immune system re-encounters the specific antigen. A hallmark of memory B cells is to display and secrete antibodies with a markedly higher affinity than those produced by primary plasma cells. During any repeat exposure the follicular helper T cell causes the memory cell to differentiate into a plasma B cell that has a greater sensitivity to that specific antigen. This jump-starts the immune system to mount a quicker, more powerful response than was possible previously. In humans, memory B cells are commonly identified by expression of CD27, coupled with low level expression of CD23/Fc epsilon RI or lack of expression of the plasma cell marker, Syndecan-1/CD138. DEP-1/CD148 is also frequently used to identify human memory B cells, as are high level expression of B7-1/CD80, B7-2/CD86, and CD95. Different subsets of memory B cells and plasma cells can be identified based on their expression of Ig isotypes (IgM, IgD, IgG, IgA), which is well understood by one of skill in the art.

In various embodiments, the iPSCs are prepared by reprogramming somatic cells. The term “induced pluripotent stem cell” or “iPSC” refers to cells derived from somatic cells, such as skin or blood cells that have been reprogrammed back into an embryonic-like pluripotent state. In some embodiments, iPSCs are generated from somatic cells such as (but not limited to) fibroblasts or PBMCs (or cells isolated therefrom). In some embodiments, the iPSCs are derived from B lymphocytes, cord blood cells (e.g., CD34+ cells), PBMCs or fraction thereof, CD34+ cells, or other human primary tissues. In some embodiments, iPSCs are derived from CD34+ cells isolated from peripheral blood. In various embodiments, the iPSCs are autologous or allogenic (e.g., HLA-matched at one or more loci) with respect to a recipient (a subject in need of treatment as described herein). In various embodiments, the iPSCs can be gene edited to assist in HLA matching (such as deletion of one or more HLA Class I and/or Class II alleles or their master regulators, including but not limited beta-2-microglobulin (B2M), CIITA, etc.), or gene edited to delete or express other functionalities. For example, iPSCs can be gene edited to delete one or more of HLA-A, HLA-B, and HLA-C, and to delete one or more of HLA-DP, HLA-DQ, and HLA-DR. In certain embodiments, the iPSCs retain expression of at least one HLA Class I and at least one HLA Class II complex. In certain embodiments, iPSCs are homozygous for at least one retained Class I and Class II loci. In some embodiments, iPSCs are prepared from B cells or other cells encoding a defined BCR or antibody having a predetermined antigen specificity (e.g., against an antigen of an infectious disease, such as a bacterial or virus surface protein). iPSCs prepared from such B cells, when differentiated to a B cell lineage, will produce B cells with the defined antigen specificity.

In various embodiments, the iPSCs are gene edited to be one of: (i) HLA-A−B+C+DP−DR+DQ+, (ii) HLA-A−B+C+DP+DR+DQ−, (iii) HLA-A−B+C+DP−DR+DQ−; (iv) HLA-A−B−C+DP−DR+DQ+; (v) HLA-A−B−C+DP+DR+DQ−, (vi) HLA-A−B−C+DP−DR+DQ−. For retained HLA (for example HLA-B, HLA-C, and HLA-DR), cells can be homozygous or retain only a single copy of the gene. For example, the modified cells are identified at least as (a) HLA-C+ and HLA-DR+, and optionally identified as one or more of (b) HLA-B−, (c) HLA-DP−, and (d) HLA-DQ−. In exemplary embodiments, the modified cells are HLA-B+, HLA-DP−, and HLA-DQ−.

In some embodiments, the iPSCs are gene edited to be HLA-A^{neg}, are homozygous for both HLA-B and HLA-C, and gene edited to be HLA-DPB1^{neg} and HLA-DQB1^{neg}. In some embodiments, the iPSCs are further homozygous for HLA-DRB1.

As used herein, the term “neg,” (−), or “negative,” with respect to a particular HLA Class I or Class II molecule indicates that both copies of the gene have been disrupted in the cell line or population, and thus the cell line or population does not display significant functional expression of the gene. Such cells can be generated by full or partial gene deletions or disruptions, or alternatively with other technologies such as siRNA. As used herein, the term “delete” in the context of a genetic modification of a target gene (i.e., gene edit) refers to abrogation of functional expression of the corresponding gene product (i.e., the corresponding polypeptide). Such gene edits include full or partial gene deletions or disruptions of the coding sequence, or deletions of critical cis-acting expression control sequences.

Somatic cells may be reprogrammed by expression of reprogramming factors selected from Sox2, Oct3/4, c-Myc, Nanog, Lin28, and klf4. In some embodiments, the reprogramming factors are Sox2, Oct3/4, c-Myc, Nanog, Lin28, and klf4. In some embodiments, the reprogramming factors are Sox2, Oct3/4, c-Myc, and klf4. Methods for preparing iPSCs are described, for example, in U.S. Pat. Nos. 10,676,165; 9,580,689; and 9,376,664, which are hereby incorporated by reference in their entireties. In various embodiments, reprogramming factors are expressed using well known viral vector systems, such as lentiviral, Sendai, or measles viral systems. Alternatively, reprogramming factors can be expressed by introducing mRNA(s) encoding the reprogramming factors into the somatic cells. Further still, iPSCs may be created by introducing a non-integrating episomal plasmid expressing the reprogramming factors, i.e., for the creation of transgene-free and virus-free iPSCs. Known episomal plasmids can be employed with limited replication capabilities and which are therefore lost over several cell generations.

In some embodiments, the human pluripotent stem cells (e.g., iPSCs) are gene-edited. Gene-editing can include, but is not limited to, modification of HLA genes (e.g., deletion of one or more HLA Class I and/or Class II genes), deletion of β2 microglobulin (β2M), deletion of CIITA, deletion or addition of B-cell receptor genes, or addition of a chimeric antigen receptor (CAR) gene, for example. An exemplary CAR-B cell can be tissue specific for inflamed or infected tissues or can be specific for target pathogens or cells. For example, the iPSCs can be B-cell receptor-transduced iPSCs. Such embodiments enable the production of large-scale regenerated B lymphocytes with a desired antigen-specificity. Alternatively, engineered iPSCs with one or more HLA knockouts can be placed in a bioreactor for a feeder-and-serum-free differentiation, under GMP-grade conditions, to generate fully functional B cells (e.g., TrB cells, Bregs, plasma B cells, memory B cells, or B cell progenitors).

In some embodiments, the iPSCs are gene edited using gRNAs that are 16, 17, 18, 19, 20, 21, 22, 23, 24, 25, 26 or more nucleotides in length. In some embodiments, the gRNAs comprise a modification at or near the 5′ end (e.g., within 1-10, 1-5, or 1-2 nucleotides of the 5′ end) and/or a modification at or near the 3′ end (e.g., within 1-10, 1-5, or 1-2 nucleotides of the 3′ end). In some embodiments, the modified gRNAs exhibit increased resistance to nucleases. In some embodiments, a gRNA comprises two separate RNA molecules (i.e., a “dual gRNA”). A dual gRNA comprises two separate RNA molecules: a “crispr RNA” (or “crRNA”) and a “tracr RNA” and is well known to one of skill in the art.

Generally, various gene editing technologies are known, which can be applied according to various embodiments of this disclosure. Gene editing technologies include but are not limited to zinc fingers (ZFs), transcription activator-like effectors (TALEs), etc. Fusion proteins containing one or more of these DNA-binding domains and the cleavage domain of Fokl endonuclease can be used to create a double-strand break in a desired region of DNA in a cell (See, e.g., US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). In some embodiments, gene editing is conducted using CRISPR associated Cas system (e.g., CRISPR-Cas9), as known in the art. See, for example, U.S. Pat. Nos. 8,697,359, 8,906,616, and 8,999,641, each of which is hereby incorporated by reference in its entirety. In various embodiments, the gene editing employs a Type II Cas endonuclease (such as Cas9) or employs a Type V Cas endonuclease (such as Cas12a). Type II and Type V Cas endonucleases are guide RNA directed. Design of gRNAs to guide the desired gene edit (while limiting or avoiding off target edits) is known in the art. See, for example, Mohr S E, et al., CRISPR guide RNA design for research applications, FEBS J. 2016 September; 283(17): 3232-3238. In still other embodiments, non-canonical Type II or Type V Cas endonucleases having homology (albeit low primary sequence homology) to S. pyogenes Cas9 or Prevotella and Francisella1 (Cpf1 or Cas12a) can be employed. Numerous such non-canonical Cas endonucleases are known in the art. Nidhi S, et al. Novel CRISPR-Cas Systems: An Updated Review of the Current Achievements, Applications, and Future Research Perspectives, Int J Mol Sci. 2021 April; 22(7): 3327. In still other embodiments, the gene editing employs base editing or prime editing to incorporate mutations without instituting double strand breaks. See, for example, Antoniou P, et al., Base and Prime Editing Technologies for Blood Disorders, Front. Genome Ed., 28 Jan. 2021; Matsuokas I G, Prime Editing: Genome Editing for Rare Genetic Diseases Without Double-Strand Breaks or Donor DNA, Front. Genet., 9 Jun. 2020. Various other gene editing processes are known, including use of dead Cas (dCas) systems (e.g., Cas fusion proteins) to target DNA modifying enzymes to desired targets using the dCas as a guide RNA-directed system. Brezgin S, Dead Cas Systems: Types, Principles, and Applications, Int J Mol Sci. 2019 December; 20(23): 6041.

Base editors that can install precise genomic alterations without creating double-strand DNA breaks can also be used in gene editing (e.g., designing gene therapy vectors) in the cells (e.g., iPSCs). Base editors essentially comprise a catalytically disabled nuclease, such as Cas9 nickase (nCas9), which is incapable of making DSBs and is fused to a nucleobase deaminase enzyme and, in some cases, a DNA glycosylase inhibitor. Currently, there are 2 major categories of base editors, cytidine base editors (CBEs) and adenine base editors (ABEs), which catalyze C>T and A>G transitions. Base editors can be delivered, for example, via HDAd5/35++ vectors to efficiently edit promoters and enhancers to active or inactivate a gene. Exemplary methods are described in U.S. Pat. Nos. 9,840,699; 10,167,457; 10,113,163; 11,306,324; 11,268,082; 11,319,532; and 11,155,803. Also contemplated are prime editors that comprise a reverse transcriptase conjugated to (e.g., fused with) a Cas endonuclease and a polynucleotide useful as a DNA synthesis template conjugated to (e.g., fused with) a guide RNA, as described in WO 2020/191153.

Exemplary vectors that can be used for the genome editing applications include, but are not limited to, plasmids, retroviral vectors, lentiviral vectors, adenovirus vectors (e.g., Ad5/35, Ad5, Ad26, Ad34, Ad35, Ad48, parvovirus (e.g., adeno-associated virus (AAV) vectors, herpes simplex virus vectors, baculoviral vectors, coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including herpes virus (e.g., Herpes Simplex virus types 1 and 2, Epstein-Barr virus, cytomegalovirus), and poxvirus (e.g., canarypox, vaccinia or modified vaccinia virus. The vector comprising the nucleic acid molecule of interest may be delivered to the cell (e.g., iPS cells, endothelial cells, hemogenic endothelial cells, HSCs (ST-HSCs or LT-HSCs) via any method known in the art, including but not limited to transduction, transfection, infection, and electroporation. Any of these vectors may include transposable element (such as a piggyback transposon or sleeping beauty transposon). Transposons insert specific sequences of DNA into genomes of vertebrate animals. The gene of interest can be integrated into the genome of a mammalian cell by transposase-catalyzed cleavage of similar excision sites that exist within the nuclear genome of the cell.

For increased efficiency, in some embodiments, the Cas and the gRNA can be combined before being delivered into the cells. The Cas-gRNA complex is known as a ribonucleoprotein (RNP). A number of methods have been developed for direct delivery of RNPs to cells. For example, RNP can be delivered into cells in culture by lipofection or electroporation. Electroporation using a nucleofection protocol can be employed, and this procedure allows the RNP to enter the nucleus of cells quickly, so it can immediately start cutting the genome. See, for example, Zhang S, Shen J, Li D, Cheng Y. Strategies in the delivery of Cas9 ribonucleoprotein for CRISPR/Cas9 genome editing. Theranostics. 2021 Jan. 1; 11(2):614-648, hereby incorporated by reference in its entirety. In some embodiments, Cas9 and gRNA are electroporated as RNP into the donor iPSCs and/or HSCs.

Generally, a protospacer adjacent motif (PAM) is required for a Cas nuclease to cut and is generally found 3-4 nucleotides downstream from the cut site. The PAM is a short DNA sequence (usually 2-6 base pairs in length) that follows the DNA region targeted for cleavage by the CRISPR system, such as CRISPR-Cas9. In some embodiments, the PAM sequences, sgRNAs, or base editing tools targeting haplotypes or polymorphs of HLA loci does not include four Gs, four Cs, GC repeats, or combinations thereof.

In some embodiments, a CRISPR/Cas9 system specific to a unique HLA haplotype can be developed by designing singular gRNAs targeting each of the donor-specific HLA-A, HLA-DPB1, and HLA-DQB1 genes (for example), using the gRNAs as described herein. To perform genetic knockout, the gRNA targets the Cas9 protein to the appropriate site to edit. Next, the Cas9 protein can perform a double strand break (DSB), where the DNA repairs through a non-homologous end joining (NHEJ) mechanism which generates indels resulting in a frameshift mutation and terminates the resulting protein's function. However, off-target genetic modifications can occur and alter the function of otherwise intact genes. For example, the Cas9 endonuclease can create DSBs at undesired off-target locations, even in the presence of some degree of mismatch. This off-target activity can create genome instability events, such as point mutations and genomic structural variations. In various embodiments, a sgRNA targeting HLA-A can target a region of chromosome 6 defined as 29942532-29942626. In various embodiments, a sgRNA targeting HLA-DQB1 can target a region of chromosome 6 defined as 32665067-32664798. In various embodiments, a sgRNA targeting HLA-DPB1 can target a region of chromosome 6 defined as 33080672-33080935.

gRNAs can be used to develop clonal iPSCs. Such iPSC lines can be evaluated for (i) ON-target edits, (ii) OFF-target edits, and (iii) Translocation edits, for example using sequencing, as described herein. Specifically, such assays can be performed by multiplex PCR with primers designed to target and enrich regions of interest followed by next-generation sequencing (e.g., Amplicon sequencing, AMP-seq). The ON-target panel and the translocation panel can amplify the intended edited region, allowing for selection of iPSC clones with the expected edits which are free from chromosomal translocation arising from unintended DSB cut-site fusion. The OFF-target panel can enrich any potential off-target regions identified via sequencing and allows for selection of iPSC clones with negligible off-target mutations. Together, these assays enable a screen of the iPSC clones to select the clones with the desired edits, while excluding potential CRISPR/Cas9-related genome integrity issues.

In some embodiments, to further ensure the genomic stability and integrity of reprogrammed and edited iPSCs, genetic and genomic assays can be performed to select for clones which, for example, did not undergo translocation and mutation events, and that did not integrate the episomal vectors. For example, whole-genome sequencing (WGS) is performed on CD34+ cells and on iPSC clones after reprogramming, where the genomes are compared for differences arising from editing. These analyses provide an assessment of which iPSC clone genomes differ from the CD34+ starting material, enabling informed selection iPSC clones which did not accrue mutations during the reprogramming.

In some embodiments, karyotyping analyses using systems such as KARYOSTAT assays is used to select iPSC clones which did not accrue indels and translocation during the reprogramming, for example as described in Ramme A P, et al, “Supporting dataset of two integration-free induced pluripotent stem cell lines from related human donors,” Data Brief. 2021 May 15; 37:107140, hereby incorporated by reference in its entirety. KARYOSTAT assays allow for visualization of chromosome aberrations with a resolution similar to G-banding karyotyping. The size of structural aberration that can be detected is >2 Mb for chromosomal gains and >1 Mb for chromosomal losses. The KARYOSTAT array is functionalized for balanced whole-genome coverage with a low-resolution DNA copy number analysis, where the assay covers all 36,000 RefSeq genes, including 14,000 OMIM targets. The assay enables the detection of aneuploidies, submicroscopic aberrations, and mosaic events.

In some embodiments, Array Comparative Genomic Hybridization (aCGH) analyses is used to select iPSC clones which did not accrue copy number aberrations (CNA) during reprogramming, for example as described in Wiesner et al. “Molecular Techniques,” Editor(s): Klaus J. Busam, Pedram Gerami, Richard A. Scolyer, “Pathology of Melanocytic Tumors,” Elsevier, 2019, pp. 364-373, ISBN 9780323374576; and Hussein S M, et al. “Copy number variation and selection during reprogramming to pluripotency,” Nature. 2011 Mar. 3; 471(7336):58-62, hereby incorporated by reference in its entirety. aCGH is a technique that analyzes the entire genome for CNA by comparing the sample DNA to reference DNA.

In some embodiments, targeted heme malignancy NGS panel analyses is used to select iPSC clones which did not accrue hematologic malignancy mutations during reprogramming. For example, targeted heme malignancy NGS panels can focus on myeloid leukemia, lymphoma, and/or other hematologic malignancy-associated genes to generate a smaller, more manageable data set than broader methods. Targeted heme malignancy NGS panel analysis includes the use of highly multiplexed PCR to amplify regions associated with hematologic malignancies followed by next-generation sequencing.

In some embodiments, Droplet Digital PCR (ddPCR) is used to select iPSC clones which did not integrate episomal vectors and that have been passaged enough for episomal vector clearance. As discussed herein, iPSC reprogramming of CD34+ cells can be achieved by delivering episomal vectors encoding reprogramming factors. However, episomal vectors can, albeit rarely, randomly integrate into the cellular genome, which could disrupt developmental processes, homeostasis, etc. Therefore, ddPCR methods can be used to detect residual episomal vector in the iPSC cultures and enable selection of iPSC clones which did not integrate episomal vectors.

In some embodiments, after assessing that the selected clones are free from genomic aberrations related to editing, the clones can be additionally tested for spontaneous mutations that might arise during expansion. For example, mutations affecting hematologic malignancy genes, indel, translocations, number aberrations, e.g., as described for the pre-edited reprogrammed clones. Analyses for spontaneous mutations can include whole-genome sequencing (WGS), KARYOSTAT analysis, Array Comparative Genomic Hybridization (aCGH) analysis, targeted heme malignancy NGS panel AMP-Seq analysis, and/or Droplet Digital PCR (ddPCR).

In various embodiments, iPSCs are prepared, and expanded using a culture system. Expanded iPSCs can be used for generating embryoid bodies (EBs). EBs, created by differentiation of iPSCs, are three-dimensional aggregates of iPSCs and comprise the three (or alternatively two or one) embryonic germ layer(s) based on the differentiation method(s). Preparation of EBs is described, for example, in US 2019/0177695, which is hereby incorporated by reference in its entirety. In some embodiments, EBs prepared by differentiation of the iPSCs, are expanded in a bioreactor as described, for example, in Abecasis B. et al., Expansion of 3D human induced pluripotent stem cell aggregates in bioreactors: Bioprocess intensification and scaling-up approaches. J. of Biotechnol. 246 (2017) 81-93. Other methods, including a 3D suspension culture, for expansion or differentiation of EBs is described in WO 2020/086889, which is hereby incorporated by reference in its entirety.

Accordance with the disclosure, CD34+ cells are isolated from the pluripotent stem cells (e.g., EBs) and endothelial-to-hematopoietic differentiation is induced to prepare a hematopoietic stem cell (HSC) population. HSCs comprising relatively high frequency of LT-HSCs can be generated from the cell populations using various stimuli or factors, including mechanical, biochemical, metabolic, and/or topographical stimuli, as well as factors such as extracellular matrix, niche factors, cell-extrinsic factors, induction of cell-intrinsic properties; and including pharmacological and/or genetic means.

In some embodiments, the method comprises preparing endothelial cells with hemogenic potential from pluripotent stem cells, prior to induction of EHT. In some embodiments, the combined over-expression of GATA2/ETV2, GATA2/TAL1, or ER71/GATA2/SCL can lead to the formation of endothelial cells with hemogenic potential from PSC sources. In some embodiments, the method comprises overexpression of E26 transformation-specific variant 2 (ETV2) transcription factor in the iPSCs. ETV2 can be expressed by introduction of an encoding non-integrating episomal plasmid, for constitutive or inducible expression of ETV2, and for production of transgene-free hemogenic ECs. In some embodiments, ETV2 is expressed from an mRNA introduced into the iPSCs. mRNA can be introduced using any available method, including electroporation or lipofection. Differentiation of cells expressing ETV2 can comprise addition of VEGF-A. See, Wang K, et al., Robust differentiation of human pluripotent stem cells into endothelial cells via temporal modulation of ETV2 with mRNA. Sci. Adv. Vol. 6 (2020). Cells generated in this manner may be used for producing CD34+ cells and inducing EHT according to embodiments of this disclosure.

Following CD34+ enrichment, HSCs are then generated from the endothelial cells using mechanical, biochemical, pharmacological and/or genetic stimulation or modification.

In some embodiments, iPSC differentiation proceeds until cells are at least about 10% CD34+, or at least about 20% CD34+, or at least about 25% CD34+, or at least about 30% CD34+. In some embodiments, CD34 enrichment and EHT may be induced at Day 7 to Day 14 of iPSC differentiation, such as for example, Day 8, Day 9, Day 10, Day 11, Day 12, Day 13, or Day 14. Differentiation of iPSCs can be according to known techniques. In some embodiments, iPSC differentiation involves factors such as, but not limited to, combinations of bFGF, Y27632, BMP4, VEGF, SCF, EPO, TPO, IL-6, IL-11, and/or IGF-1. In some embodiments, hPSCs are differentiated using feeder-free, serum-free, and/or GMP-compatible materials. Serum free culture generally comprises a cocktail of cytokines/growth factors/small molecules.

In a non-limiting example, isolated iPSCs can be cultured under conditions that promote lympho-hematopoiesis. In some embodiments, feeder cells, such as a feeder layer of STO mouse fibroblast cells, can be used to expand the B cells. In some embodiments, hPSCs are co-cultured with murine bone marrow-derived feeder cells such as OP9 or blood-derived peripheral blood mononuclear cells (PBMCs) or cord blood-derived mesenchymal stem cells or lymphocyte-derived cancer cell lines cells in serum-containing medium. The culture can contain growth factors and cytokines to support differentiation of embryoid bodies or monolayer system. The feeder cell co-culture system can be used to generate multipotent HSPCs, which can be differentiated further to several hematopoietic lineages including B lymphocytes, monocytes or macrophages, dendritic cells, neutrophils, NK cells, T lymphocytes, megakaryocytes, and erythrocytes. See Netsrithong R. et al., Multilineage differentiation potential of hematoendothelial progenitors derived from human induced pluripotent stem cells, Stem Cell Research & Therapy Vol. 11 Art. 481 (2020). Alternatively, a stepwise process using defined conditions with specific signals can be used. For example, the expression of HOXA9, ERG, RORA, SOX4, and MYB in human PSCs favors the direct differentiation into CD34+/CD45+ progenitors with multilineage potential. Further, expression of factors such as HOXB4, CDX4, SCL/TAL1, or RUNX1a support the hematopoietic program in human PSCs. See Doulatov S. et al., Induction of multipotential hematopoietic progenitors from human pluripotent stem cells via re-specification of lineage-restricted precursors, Cell Stem Cell. 2013 Oct. 3; 13(4).

Differentiation of iPSCs (e.g., to EBs) may employ a WNT agonist, such as CHIR99021. A WNT agonist is a molecule that mimics or increases WNT signaling. Non-limiting examples of WNT agonists include small molecules CHIR-99021 (CAS 252917-06-9), a 2-amino-4,6-disubstituted pyrimidine, e.g. BML 284 (CAS 853220-52-7), SKL 2001 (CAS 909089-13-0), WAY 262611 (CAS 1123231-07-1), WAY 316606 (CAS 915759-45-4), SB 216763 (CAS 280744-09-4), IQ 1 (CAS 331001-62-8), QS 11 (CAS 944328-88-5), deoxycholic acid (CAS 83-44-3), BIO (CAS 667463-62-9), kenpaullone (CAS 142273-20-9), or a (hetero) arylpyrimidine. In some embodiments, a WNT agonist is an agonist antibody or functional fragment thereof or an antibody-like polypeptide.

Differentiation of iPSCs (e.g., to EBs) may employ a ROCK inhibitor. Exemplary ROCK inhibitors that find use for establishing and differentiation iPSCs include but are not limited to: thiazovivin, Y27632, Fasudil, AR122-86, RevitaCell™ Supplement, H-1152, Y-30141, Wf-536, HA-1077, hydroxyl-HA-1077, GSK269962A, SB-772077-B, N-(4-Pyridyl)-N′-(2,4,6-trichlorophenyl)urea, 3-(4-Pyridyl)-1H-indole, and (R)-(+)-trans-N-(4-Pyridyl)-4-(1-aminoethyl)-cyclohexanecarboxamide, H-100, and ROCK inhibitors disclosed in U.S. Pat. No. 8,044,201, which is hereby incorporated by reference in its entirety.

Induction of EHT can be with any known process. In some embodiments, induction of EHT generates a hematopoietic stem cell (HSC) population comprising LT-HSCs. In some embodiments, EHT generates HSCs through endothelial or hemogenic endothelial cell (HEC) precursors using mechanical, biochemical, pharmacological and/or genetic means (e.g., via stimulation, inhibition, and/or genetic modifications). In some embodiments, the EHT generates a stem cell population comprising one or more of long-term hematopoietic stem cells (LT-HSCs), short-term hematopoietic stem cells (ST-HSCs), and hematopoietic stem progenitor cells. In some embodiments, the EHT culture includes one or more (e.g., combinations of) Y-27632, TPO, IL-3, SCF, IL-6, IL-11, IGF-1, VEGF, bFGF, BMP4, and FLT3.

In some embodiments, the method comprises increasing the expression or activity of dnmt3b in PSCs, embryoid bodies, CD34+-enriched cells, ECs, HECs or HSCs, which can be by mechanical, genetic, biochemical, or pharmacological means. In some embodiments, the method comprises increasing activity or expression of DNA (cytosine-5-)-methyltransferase 3 beta (Dnmt3b) and/or GTPase IMAP Family Member 6 (Gimap6) in the cells. See WO 2019/236943 and WO 2021/119061, which are hereby incorporated by reference in their entirety. In some embodiments, the induction of EHT comprises increasing the expression or activity of dnmt3b.

In some embodiments, cells are contacted with an effective amount of an agonist of a mechanosensitive receptor or a mechanosensitive channel that increases the activity or expression of Dnmt3b. In some embodiments, the mechanosensitive receptor is Piezol. An exemplary Piezol agonist is Yoda1. In some embodiments, the mechanosensitive receptor is Trpv4. An exemplary Trpv4 agonist is GSK1016790A. Yoda1 (2-[5-[[(2,6-Dichlorophenyl)methyl]thio]-1,3,4-thiadiazol-2-yl]-pyrazine) is a small molecule agonist developed for the mechanosensitive ion channel Piezol. Syeda R, Chemical activation of the mechanotransduction channel Piezol. eLife (2015).

Derivatives of Yoda1 can be employed in various embodiments. For example, derivatives comprising a 2,6-dichlorophenyl core are employed in some embodiments. Exemplary agonists are disclosed in Evans E L, et al., Yoda1 analogue (Dooku1) which antagonizes Yoda1-evoked activation of Piezol and aortic relaxation, British J. of Pharmacology 175(1744-1759): 2018. Still other Piezol agonist include Jedi1, Jedi2, single-stranded (ss) RNA (e.g., ssRNA40) and derivatives and analogues thereof. See Wang Y., et al., A lever-like transduction pathway for long-distance chemical- and mechano-gating of the mechanosensitive Piezol channel. Nature Communications (2018) 9:1300; Sugisawa, et al., RNA Sensing by Gut Piezol Is Essential for Systemic Serotonin Synthesis, Cell, Volume 182, Issue 3, 2020, Pages 609-624, incorporated herein in their entirety by reference. These Piezol agonists are commercially available. In various embodiments, the effective amount of the Piezol agonist or derivative is in the range of about 1 μM to about 500 μM, or about 5 μM to about 200 μM, or about 5 μM to about 100 μM, or in some embodiments, in the range of about 25 μM to about 150 μM, or about 25 μM to about 100 PM, or about 25 μM to about 5 μM.

In various embodiments, pharmacological Piezol activation is applied to CD34+ cells (i.e., CD34+-enriched cells). In certain embodiments, pharmacological Piezol activation may further be applied to iPSCs, embryoid bodies, ECs, hemogenic endothelial cells (HECs), HSCs, hematopoietic progenitors, as well as hematopoietic lineage(s). In certain embodiments, Piezol activation is applied at least to EBs generated from iPSCs, CD34+ cells isolated from EBs, and/or combinations thereof, which in accordance with various embodiments, allows for superior generation of B cell lineage cells as compared to other methods for inducing EHT. In a non-limiting example, EHT CD34+ cells are treated with Yoda 1 for 2-7 days or 3-7 days or 4 to 7 days or 5-7 days or 6-7 days, under culture conditions, to generate superior HSCs with a greater potential to generate B cell lineages or their progenitors.

Alternatively or in addition, the activity or expression of Dnmt3b can be increased directly in the cells, e.g., in CD34+-enriched cells. For example, mRNA expression of Dnmt3b can be increased by delivering Dnmt3b-encoding transcripts to the cells, or by introducing a Dnmt3b-encoding transgene, or a transgene-free method, not limited to introducing a non-integrating episome to the cells. In some embodiments, gene editing is employed to introduce a genetic modification to Dnmt3b expression elements in the cells, such as, but not limited to, to increase promoter strength, ribosome binding, RNA stability, and/or impact RNA splicing.

In some embodiments, the method comprises increasing the activity or expression of Gimap6 in the cells, alone or in combination with Dnmt3b and/or other genes that are up- or down regulated upon cyclic strain or Piezol activation. To increase activity or expression of Gimap6, Gimap6-encoding mRNA transcripts can be introduced to the cells, transgene-free approaches can also be employed, including but not limited, to introducing an episome to the cells; or alternatively a Gimap6-encoding transgene. In some embodiments, gene editing is employed to introduce a genetic modification to Gimap6 expression elements in the cells (such as one or more modifications to increase promoter strength, ribosome binding, RNA stability, or to impact RNA splicing).

In embodiments of this disclosure employing mRNA delivery to cells, known chemical modifications can be used to avoid the innate-immune response in the cells. For example, synthetic RNA comprising only canonical nucleotides can bind to pattern recognition receptors, and can trigger a potent immune response in cells. This response can result in translation block, the secretion of inflammatory cytokines, and cell death. RNA comprising certain non-canonical nucleotides can evade detection by the innate immune system, and can be translated at high efficiency into protein. See U.S. Pat. No. 9,181,319, which is hereby incorporated by reference, particularly with regard to nucleotide modification to avoid an innate immune response.

In some embodiments, expression of Dnmt3b and/or Gimap6 is increased by introducing a transgene into the cells, which can direct a desired level of overexpression (with various promoter strengths or other selection of expression control elements). Transgenes can be introduced using various viral vectors or transfection reagents (including 5 Lipid Nanoparticles) as are known in the art. In some embodiments, expression of Dnmt3b and/or Gimap6 is increased by a transgene-free method (e.g., episome delivery). In some embodiments, expression or activity of Dnmt3b and/or Gimap6 or other genes disclosed herein are increased using a gene editing technology, for example, to introduce one or more modifications to increase promoter strength, ribosome binding, or RNA stability.

In some embodiments, the method comprises applying cyclic 2D, 3D, or 4D stretch to cells. In various embodiments, the cells subjected to cyclic 2D, 3D, or 4D stretch are selected from one or more of CD34-enriched cells, iPSCs, ECs, and HECs. For example, a cell population is introduced to a bioreactor that provides a cyclic-strain biomechanical stretching, as described in WO 2017/096215, which is hereby incorporated by reference in its entirety. The cyclic-strain biomechanical stretching can increase the activity or expression of Dnmt3b and/or Gimap6. In these embodiments, mechanical means apply stretching forces to the cells, or to a cell culture surface having the cells (e.g., ECs or HECs) cultured thereon. For example, a computer controlled vacuum pump system or other means for providing a stretching force (e.g., the FlexCell™ Tension System, the Cytostretcher System) attached to flexible biocompatible and/or biomimetic surface can be used to apply cyclic 2D, 3D, or 4D stretch ex vivo to cells under defined and controlled cyclic strain conditions. For example, the applied cyclic stretch can be from about 1% to about 20% cyclic strain (e.g., about 6% cyclic strain) for several hours or days (e.g., about 7 days). In various embodiments, cyclic strain is applied for at least about one hour, at least about two hours, at least about six hours, at least about eight hours, at least about 12 hours, at least about 24 hours, at least about 48 hrs, at least about 72 hrs, at least about 96 hrs, at least about 120 hrs, at least about 144 hrs, or at least about 168 hrs.

Alternatively or in addition, EHT is stimulated by Trpv4 activation. The Trpv4 activation can be by contacting cells (e.g., CD34-enriched cells, ECs, or HECs) with one or more Trpv4 agonists, which are optionally selected from GSK1016790A, 4alpha-PDD, or analogues and/or derivatives thereof.

Where cell populations are described herein as having a certain phenotype it is understood that the phenotype represents a significant portion of the cell population, such as at least 25%, at least 40%, or at least about 50%, or at least about 60%, or at least about 75%, or at least about 80%, or at least about 90% of the cell population. Further, at various steps, cell populations can be enriched for cells of a desired phenotype, and/or depleted of cells of an undesired phenotype, such that cell population comprise at least about 75%, or at least about 80%, or at least about 90% of the desired phenotype. Such positive and negative selection methods are known in the art. For example, cells can be sorted based on cell surface antigens (including those described herein) using a fluorescence activated cell sorter, or magnetic beads which bind cells with certain cell surface antigens. Negative selection columns can be used to remove cells expressing undesired cell-surface markers. In some embodiments, cells are enriched for CD34+ cells (prior to and/or after undergoing EHT). In some embodiments, the cell population is cultured under conditions that promote expansion of CD34+ cells to thereby produce an expanded population of stem cells. Further, subpopulations of B cell lineages may be enriched or isolated using these techniques.

In various embodiments, CD34+ cells (e.g., the floater and/or adherent cells) are harvested from the culture undergoing endothelial-to-hematopoietic transition between Day 8 to Day 20 of iPSC differentiation (such as from Day 10 to Day 17 of iPSC differentiation).

In various embodiments, the HSCs or CD34-enriched cells are further expanded. For example, the HSCs or CD34-enriched cells can be expanded according to methods disclosed in U.S. Pat. Nos. 8,168,428; 9,028,811; 10,272,110; and 10,278,990, which are hereby incorporated by reference in their entireties. In some embodiments, ex vivo expansion of HSCs or CD34-enriched cells employs prostaglandin E2 (PGE2) or a PGE2 derivative. In some embodiments of this disclosure, the HSCs comprise at least about 0.01% LT-HSCs, or at least about 0.05% LT-HSCs, or at least about 0.1% LT-HSCs, or at least about 0.5% LT-HSCs, or at least about 1% LT-HSCs.

Hematopoietic stem cells (HSCs) which promote lympho-hematopoiesis or give rise to lymphoid lineages, can be identified based on the expression of CD34 and the absence of lineage specific markers (termed Lin-). In some embodiments, a population of stem cells comprising HSCs are enriched, for example, as described in U.S. Pat. No. 9,834,754, which is hereby incorporated by reference in its entirety. For example, this process can comprise sorting a cell population based on expression of one or more of CD34, CD90, CD38, CD43, CD45, CD19, CD20, CD138 or CD10. A fraction can be selected for further differentiation that is one or more of CD34⁺, CD90⁺, CD38⁻, CD19, CD20, CD138 and/or CD43. In some embodiments, the stem cell population for differentiation to a hematopoietic lineage is at least about 80% CD34⁺, or at least about 90% CD34⁺, or at least about 95% CD34⁺.

In some embodiments, the stem cell population, or CD34+-enriched cells or fraction thereof, or derivative population are expanded as described in US 2020/0308540, which is hereby incorporated by reference in its entirety. For example, the cells are expanded by exposing the cells to an aryl hydrocarbon receptor antagonist including, for example, SR1 or an SR1-derivative. See also, Wagner et al., Cell Stem Cell 2016; 18(1):144-55 and Boitano A., et al., Aryl Hydrocarbon Receptor Antagonists Promote the Expansion of Human Hematopoietic Stem Cells. Science 2010 Sep. 10; 329(5997): 1345-1348.

In some embodiments, the compound that promotes expansion of CD34⁺ cells includes a pyrimidoindole derivative including, for example, UM171 or UM729 (see US 2020/0308540, which is hereby incorporated by reference).

In some embodiments, the stem cell population or CD34+-enriched cells are further enriched for cells that express Periostin and/or Platelet Derived Growth Factor Receptor Alpha (pdgfra) or are modified to express Periostin and/or pdgfra, as described in WO 2020/205969 (which is hereby incorporated by reference in its entirety). Such expression can be by delivering encoding transcripts to the cells, or by introducing an encoding transgene, or a transgene-free method, not limited to introducing a non-integrating episome to the cells. In some embodiments, gene editing is employed to introduce a genetic modification to expression elements in the cells, such as to modify promoter activity or strength, ribosome binding, RNA stability, or impact RNA splicing.

In still other embodiments, the stem cell population or CD34+-enriched cells are cultured with an inhibitor of histone methyltransferase EZH1. Alternatively, EZH1 is partially or completely deleted or inactivated or is transiently silenced in the stem cell population. Inhibition of EZH1 can direct myeloid progenitor cells (e.g., CD34+CD45+) to lymphoid lineages. See WO 2018/048828, which is hereby incorporated by reference in its entirety. In still other embodiments, EZH1 is overexpressed in the stem cell population.

In various embodiments, the HSC population or fraction thereof is differentiated to a B cell lineage or a progenitor thereof, such as, multipotent progenitor cells (MPPs), common lymphoid precursor (CLP), common lymphoid 2 progenitor (LCA-2), early pro-B cells, late pro B cells, pre B cell, immature B cell lineages and fractions thereof, from which mature B cells can be generated. B cells generated according to this process may have phenotypes consistent with transitional B cells, regulatory B cells, marginal zone B cells, follicular B cells, activated B cells, memory B cells or plasma B cells, as already described.

In some embodiments, the HSC population or fraction thereof is differentiated to B cells or progenitors or derivatives thereof independent of the use of an agonist of a mechanosensitive receptor or a mechanosensitive channel such as Yoda1. In some embodiments, the use of an agonist of a mechanosensitive receptor or a mechanosensitive channel such as Yoda1 is optional. Thus, in some embodiments, CD34+ cells are enriched from a differentiated pluripotent stem cell population to prepare a CD34+-enriched population. Endothelial-to-hematopoietic transition of the CD34+-enriched cell population is induced for at least two days, but no more than 12 days in which the use of an agonist of a mechanosensitive receptor or a mechanosensitive channel such as Yoda1, jedi1, jedi2, ssRNA40 is optional. The HSCs and/or HSPCs are differentiated to a progenitor B cell population or a B cell population.

In some embodiments, the endothelial-to-hematopoietic transition of the CD34+-enriched cell population is induced for at least for two days and optionally further for at least about 4 hours, or at least about 8 hours, or at least about 12 hours, or at least about 16 hours, or at least about 20 hours, or at least about 24 hours, or at least about 2 days, or at least about 3 days, or at least about 4 days, or at least about 5 days, or at least about 6 days, or at least about 7 days, or at least about 8 days, or at least about 9 days, or at least about 10 days. Generally, EHT is not induced for more than 12 days. In various embodiments, EHT is induced for 4 days to about 8 days, or from 5 days to about 7 days.

In some embodiments, lympho-hematopoietic lineage is cultured with a Notch ligand, partial or full, SHH, extracellular matrix component(s), and/or combinations thereof, ex vivo, to differentiate HSCs to lympho-hematopoietic lineage. Further, according to known processes, xenogenic OP9-DL lor a feeder layer of STO mouse fibroblasts or blood-derived peripheral blood mononuclear cells (PBMCs) or cord blood-derived mesenchymal stem cells or lymphocyte-derived cancer cell lines cells can be employed for differentiation of hematopoietic cells to lympho-hematopoietic lineage, T cells or NK cells and may optionally be employed in the differentiation of HSCs into other lineages. The OP9-DL1 co-culture system uses a bone marrow stromal cell line (OP9) transduced with the Notch ligand delta-like-1 (DLL1) to support T cell development from stem cell sources. The OP9-DL1 system limits the potential of the cells for clinical application. Thus, in some embodiments the method employs a feeder-cell-free systems for generation of B cell lineages from the hiPSCs for clinical use. In a non-limiting example, to generate lympho-hematopoietic lineage derived cells utilizing the notch ligands, iPSC expansion is performed for 6 days, followed by embryoid body formation, which takes about 8 days. The cells are further cultured for about 5 days to enable the development of CD34+ hemogenic endothelial cells, from which HSCs are derived. The HSCs are then cultured in media promoting differentiation to lympho-hematopoietic cells, such as the B cell lineages described herein.

In some embodiments, presence of cytokines and/or growth factors are desired, these include but are not limited to, stem cell factor, Fms-like tyrosine kinase 3 ligand, VEGF, bFGF, SCF, Flt3L, TPO, IL3, IL7, and IL15; and optionally, a BMP activator, to initiate differentiation of the definitive hemogenic endothelium to multipotent progenitor cells (MPPs), common lymphoid precursor (CLP), common lymphoid 2 progenitor (LCA-2), early pro-B cells, late pro B cells, pre B cell, immature B cell lineages and optionally, (ii) contacting pluripotent stem cells-derived lympho-hematopoietic lineage cells with a composition comprising one or more growth factors and cytokines selected from SCF, Flt3L, IL3, IL7, and IL15, wherein the medium is free of one or more of VEGF, bFGF, TPO, BMP activators and ROCK inhibitors, to initiate differentiation of the lympho-hematopoietic lineage.

The term “Notch ligand” as used herein refers to a ligand capable of binding to a Notch receptor polypeptide present in the membrane of a hematopoietic stem cell or progenitor T cell. The Notch receptors include Notch-1, Notch-2, Notch-3, and Notch-4. Notch ligands typically have a DSL domain (D-Delta, S-Serrate, and L-Lag2) comprising 20-22 amino acids at the amino terminus, and from 3 to 8 EGF repeats on the extracellular surface. In various embodiments, the Notch ligand comprises at least one of Delta-Like-1 (DLL1), Delta-Like-4 (DLL4), SFIP3, or a functional portion thereof, Jagged 1 (JAG1), Jagged 2 (JAG2), Delta-like ligand 3 (DLL3), and X-delta 2. A key signal that is delivered to incoming lymphocyte progenitors by the thymus stromal cells in vivo is mediated by DL4, which is expressed by cortical thymic epithelial cells.

“Notch ligand” as used herein also includes intact (full-length), partial (a truncated form), or modified (comprising one or more mutations, such as conservative mutations) notch ligands as well as Notch ligands from any species or fragments thereof that retain at least one activity or function of a full-length Notch ligand. Also included are peptides that mimic notch ligands. Notch ligands can be “canonical notch ligands” or “non-canonical notch ligands.” Canonical notch ligands are characterized by extracellular domains typically comprising an N-terminal (NT) domain followed by a Delta/Serrate/LAG-2 (DSL) domain and multiple tandemly arranged Epidermal Growth Factor (EGF)-like repeats. The DSL domain together with the flanking NT domain and the first two EGF repeats containing the Delta and OSM-11-like proteins (DOS) motif are typically required for canonical ligands to bind Notch. The intracellular domains of some canonical ligands contain a carboxy-terminal PSD-95/Dlg/ZO-1-ligand (PDZL) motif that plays a role independent of Notch signaling. C. elegans DSL ligands lack a DOS motif but have been proposed to cooperate with DOS-only containing ligands to activate Notch signaling.

In some embodiments, the Notch ligand is an anti-Notch (agonistic) antibody that can bind and engage Notch signaling. In some embodiments, the antibody is a monoclonal antibody (including a human or humanized antibody), a single chain antibody (scFv), a nanobody, or other antibody fragment or antigen-binding molecule capable of activating the Notch signaling pathway.

In some embodiments, the Notch ligand is a Delta family Notch ligand. The Delta family ligand in some embodiments is Delta-1 (Genbank Accession No. AF003522, Homo sapiens), Delta-like 1 (DLL1, Genbank Accession No. NM_005618 and NP_005609, Homo sapiens; Genbank Accession No. X80903, 148324, M. musculus), Delta-4 (Genbank Accession No. AF273454, BAB18580, Mus musculus; Genbank Accession No. AF279305, AAF81912, Homo sapiens), and/or Delta-like 4 (DLL4; Genbank Accession. No. Q9NR61, AAF76427, AF253468, NM_019074, Homo sapiens; Genbank Accession No. NM 019454, Mus musculus). Notch ligands are commercially available or can be produced, for example, by recombinant DNA techniques.

In some embodiments, the Notch ligand comprises an amino acid sequence that is at least about 70%, or at least about 80%, or at least about 90%, or at least about 95%, or at least about 97% identical (e.g., about 100% identical) to human DLL1 or DLL4 Notch ligand. Functional derivatives of Notch ligands (including fragments or portions thereof) will be capable of binding to and activating a Notch receptor. Binding to a Notch receptor may be determined by a variety of methods known in the art including in vitro binding assays and receptor activation/cell signaling assays.

In various embodiments, the HSC/HSPC population is cultured in an artificial thymic organoid (ATO). See, Hagen, M. et al. (2019). The ATO will include culture of HSCs (or aggregates of HSCs) with a Notch ligand-expressing stromal cell line in serum-free conditions. The artificial thymic organoid is a 3D system, inducing differentiation of hematopoietic precursors to naive CD3+CD8+ and CD3*CD4+ T cells or to B cell lineages.

In some embodiments, an artificial thymic organoid comprises DLL4 and BMP2, or functional fragments thereof.

In some embodiments, the Notch ligand is a DLL4 having one or more affinity enhancing mutations, such as one or more (or all) of: G28S, F107L, I143F, H194Y, L206P, N257P, T271L, F280Y, S301R and Q305P, with respect to hDLL4. See Gonzalez-Perez, et al., Affinity-matured DLL4 ligands as broad-spectrum modulators of Notch signaling, Nature Chemical Biology (2022).

In various embodiments, the Notch ligands are soluble, and are optionally immobilized on microparticles or nanoparticles, which are optionally paramagnetic to allow for magnetic enrichment or concentration processes. In still other embodiments, the Notch ligands are immobilized on a 2D or 3D culture surface, optionally with other adhesion molecules such as VCAM-1. See US 2020/0399599, which is hereby incorporated by reference in its entirety. In other embodiments, the beads or particles are polymeric (e.g., polystyrene or PLGA), gold, iron dextran, or constructed of biological materials, such as particles formed from lipids and/or proteins. In various embodiments, the particle has a diameter or largest dimension of from about 0.01 μm (10 nm) to about 500 μm (e.g., from about 1 μm to about 7 μm). In still other embodiments, polymeric scaffolds with conjugated ligands can be employed, as described in WO 2020/131582, which is hereby incorporated by reference in its entirety. For example, scaffold can be constructed of polylactic acid, polyglycolic acid, PLGA, alginate or an alginate derivative, gelatin, collagen, agarose, hyaluronic acid, poly(lysine), polyhydroxybutyrate, poly-epsilon-caprolactone, polyphosphazines, poly(vinyl alcohol), poly(alkylene oxide), poly(ethylene oxide), poly(allylamine), poly(acrylate), poly(4-aminomethylstyrene), pluronic polyol, polyoxamer, poly(uronic acid), poly(anhydride), poly(vinylpyrrolidone), and any combination thereof. In some embodiments, the scaffold comprises pores having a diameter between about 1 pm and 100 pm.

In some embodiments, the C-terminus of the Notch ligand is conjugated to the selected support. In some embodiments, this can include adding a sequence at the C-terminal end of the Notch ligand that can be enzymatically conjugated to the support, for example, through a biotin molecule. In another embodiment, a Notch ligand-Fc fusion is prepared, such that the Fc segment can be immobilized by binding to protein A or protein G that is conjugated to the support. Of course, any of the known protein conjugation methods can be employed.

Thus, in various embodiments, the Notch ligand is immobilized, functionalized, and/or embedded in 2D or 3D culture system. The Notch ligand may be incorporated along with a component of extracellular matrix, such as one or more selected from fibronectin, RetroNectin, and laminin. In some embodiments, the Notch ligand and/or component of extracellular matrix are embedded in inert materials providing 3D culture conditions. Exemplary materials include, but are not limited to, cellulose, alginate, and combinations thereof. In some embodiments, the Notch ligand, a component of extracellular matrix, or combinations thereof, are in contact with culture conditions providing topographical patterns and/or textures (e.g., roughness) to cells conducive to differentiation and/or expansion.

In the various aspects and embodiments, this disclosure provides a culture platform method for obtaining a B cell lineage (as described) or progenitor thereof. In various embodiments, the method comprises contacting the cells (e.g., CD34+ cells from EBs generated as described herein) with an effective amount of an agonist of a mechanosensitive receptor or a mechanosensitive channel that increases the activity or expression of Dnmt3b. In some embodiments, the mechanosensitive receptor is Piezol. Exemplary Piezol agonists include Yoda1, single-stranded (ss) RNA (e.g., ssRNA40) Jedi1, and Jedi2. In some embodiments, the mechanosensitive receptor is Trpv4. An exemplary Trpv4 agonist is GSK1016790A. The medium may comprise one or more of growth factors and cytokines selected from TPO, SCF, FIt3L, IL3, IL-6, IL7, IL-11, IGF, bFGF, and IL15. The medium may optionally comprise one or more of VEGF, bFGF, a BMP activator, a Wnt pathway activator, or ROCK inhibitors (e.g., thiazovivin or Y27632). HSC populations produced accordingly (and which may be enriched for CD34+ cells) are cultured in the presence of cytokines, growth factors, and/or small molecules that promote formation of lymphocytes, including B cell lineages. For example, such cytokines and growth factors can include combinations of IL-3, IL-7, IL-15, SCF, and FLT-3L. IL-3 can be excluded in some embodiments, particularly in the later stages of the culture. For example, CD19+ cells can be cultured with IL-7, SCF, and Flt3L. Other processes for generating B cell lineages are known in the art can be employed. B cells can be activated ex vivo using cytokines and factors such as IL-4, IL-5, and IL-6, and CD40 ligand.

In some embodiments, the B cell lineage expresses a chimeric antigen receptor (CAR), based on gene editing of iPSC, embryonic bodies, hCD34+ cells, or B cell progenitor or lineage. In some embodiments, the B cell lineage expresses a CAR via mRNA expression. Additionally, or optionally, the B CAR may be engineered to express cytokines (e.g., IL-4, IL-6, IL-15 etc. or an interferon) to make the B cell-CAR more potent in targeting tumors or infected cells.

In a non-limiting example, B cells or progenitors can be efficiently transduced by a vector, such as but not limited to retroviral or nonintegrating viral vectors (e.g., adenoviral, adeno-associated viral, integration-deficient retro-lentiviral, poxviral) or nonviral vectors (e.g., plasmid vectors, artificial chromosomes) or episomal or episomal hybrid vectors) carrying a first, second, third, fourth or fifth-generation CAR (see, for example, Sadelain et al., Cancer Discov. 3(4):388-398 (2013); Jensen et al., Immunol. Rev. 257: 127-133 (2014); Sharpe et al., Dis. Model Meeh. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32: 169-180 (2009)). Each of these aforementioned references are incorporated herein by reference in their entireties) targeting tumor antigen.

CARs are designed to enhance a cells ability to recognize, bind to, and kill target cells, such as tumor cells or virally infected cells or tissues. In some embodiments, the CAR enhances the B cell's ability to recognize target cells. In some embodiments, the CAR enhances the B cell's activity, for example through B-cell activation via antigen presentation, co-stimulation, and cytokine production. In some embodiments, CAR-B cells target the following tumors or tumor antigens:
(i) Human epidermal growth factor receptor 2 (HER2)—ovarian cancer, breast cancer, glioblastoma, colon cancer, osteosarcoma, and medulloblastoma;
(ii) Epidermal growth factor receptor (EGFR)—non-small cell lung cancer, epithelial carcinoma, and glioma;
(iii) Mesothelin—mesothelioma, ovarian cancer, and pancreatic adenocarcinoma;
(iv) Prostate-specific membrane antigen (PSMA)—prostate cancer;
(v) Carcinoembryonic antigen (CEA)—pancreatic adenocarcinoma, breast cancer, and colorectal carcinoma;
(vi) Glypican-3—hepatocellular carcinoma;
(vii) Variant III of the epidermal growth factor receptor (EGFRvIII)—glioblastoma;
(viii) Disialoganglioside 2 (GD2)—neuroblastoma and melanoma;
(ix) Carbonic anhydrase IX(CAIX)—renal cell carcinoma;
(x) Interleukin-13Ra2—glioma;
(xi) Fibroblast activation protein (FAP)—malignant pleural mesothelioma;
(xii) L1 cell adhesion molecule (L1-CAM)—neuroblastoma, melanoma, and ovarian;
(xiii) Cancer antigen 125 (CA 125)—epithelial ovarian cancer;
(xiv) Cluster of differentiation 133 (CD 133)—glioblastoma and cholangiocarcinoma, adenocarcinoma;
(xv) Cancer/testis antigen 1B (CTAG1B)—melanoma and ovarian cancer;
(xvi) Mucin 1—seminal vesicle cancer;
(xvii) Folate receptor-a (FR-a)—ovarian cancer;
(xviii) Growth factor receptor selected from one or more of ErbB1, ErbB2, ErbB3, or ErbB4, IGF1R, IGF2R, TβR I-II, VEGFR1, VEGFR2, VEGFR3, PDGFR (α/β) or FGFR1 through 4.

Therefore, in some aspects and embodiments of the invention, a genetically modified B cell line or a precursor or progeny thereof is engineered to express a chimeric antigen receptor (CAR) on a cell surface, and particularly a CAR that specifically binds to a growth factor receptor. Most typically, the CAR comprises an intracellular domain from the Fc epsilon receptor gamma (Fc epsilon RI gamma). However, in further contemplated embodiments the CAR may also comprise a T cell receptor (TCR) CD3 zeta (CD3zeta) intracellular domain, alone or in combination with additional components from the second or third generation CAR constructs (e.g., CD28, CD134, CD137, and/or ICOS).

In some embodiments, a CAR comprises at least one domain that inhibits anti-autoimmune or phagocytic signaling (e.g., an extracellular domain, a transmembrane domain, and/or an intracellular domain) in the B cell. In some embodiments, a CAR improves effector activity of the B cell, for example, by inhibiting CD47 and/or SIRP alpha activity, relative to a cell of the same type without the CAR. In some embodiments, the CAR serves as a dominant negative receptor by binding to CD47 and inhibiting SIRP alpha activity (e.g., a CD47 sink).

In some embodiments, the CAR-modified B cell exhibits increased production of one or more inflammatory cytokines relative to an unmodified B cell. The one or more inflammatory cytokines can be selected from one or more of TNF alpha, IL-6, IL-1a, IL-1b, IL-12, IL-18, IL-8, IL-2, IL-23, IFN alpha, IFN beta, IFN gamma, IL-2, IL-8, IL33, CCL3, CXCL12, CCL22, CCL4, CXCL10, or CCL2.

In one aspect, the present disclosure provides a cell composition comprising the cell population (e.g., B cell lineage population). The cell composition of this disclosure (e.g., prepared according to this disclosure) may further comprise a pharmaceutically acceptable excipient or a carrier. Such excipients or carrier solutions also can contain buffers, diluents, and other suitable additives. A buffer refers to a solution or liquid whose chemical makeup neutralizes acids or bases without a significant change in pH. Examples of buffers envisioned by the invention include, but are not limited to, normal/physiologic saline (0.9% NaCl), 5% dextrose in water (D5W), Dulbecco's phosphate buffered saline (PBS), Ringer's solution. The composition may comprise a vehicle suitable for intravenous infusion or other administration route, and the composition may include a suitable cryoprotectant. An exemplary carrier is DMSO (e.g., about 10% DMSO). Other carriers may include dimethoxy ethane (DME), N,N-dimethylformamide (DMF), or dimethylacetamide, including mixtures or combinations thereof. Cell compositions may be provided in implantable devices (e.g., scaffolds) or in bags or in vials, tubes or a container in an appropriate volume and stored frozen until use.

In various embodiments, the B cells are derived from HLA-edited iPSCs as described. For example, in some embodiments the B cells are HLA-A^{neg}, homozygous for both HLA-B and HLA-C, HLA-DPB1^{neg} and HLA-DQB1^{neg}. In some embodiments, the B cells are further homozygous for HLA-DRB1.

In some embodiments, the cell population is a progenitor B cell population capable of engraftment in a thymus, spleen, or secondary lymphoid organ upon administration to a subject in need. In various embodiments, the composition for cellular therapy is prepared that comprises the cell population and a pharmaceutically acceptable vehicle. The pharmaceutical composition may comprise at least about 10² cells, or at least about 10³, or at least about 10⁴, or at least about 10⁵, or at least about 10⁶, or at least about 10⁷, or at least about 10⁸ cells, or at least about 10⁹ cells, or at least about 10¹⁰ cells, or at least about 10¹¹ cells, or at least about 10¹² cells, or at least about 10¹³ cells, or at least about 10¹⁴ cells. For example, in some embodiments, the pharmaceutical composition is administered, comprising cells of from about 100,000 to about 400,000 cells per kilogram (e.g., about 200,000 cells/kg). In other embodiments, cells are administered at from about 10⁵ to about 5×10⁵ cells per kilogram of recipient body weight (e.g., about 2.5×10⁵ cells/kg), or from about 10⁶ to about 5×10⁶ cells per kilogram (e.g., about 2.5×10⁶ cells/kg), or from about 5×10⁶ to about 10⁷ cells per kilogram (e.g., about 5×10⁶ cells/kg) or from about 10⁷ to about 10⁸ cells per kilogram (e.g., about 5×10⁷ cells/kg) or from about 10⁸ to about 10⁹ cells per kilogram (e.g., about 5×10⁸ cells/kg) or from about 10⁹ to about 10¹⁰ cells per kilogram or from about 10¹⁰ to about 10¹¹ cells or from about 10¹¹ to about 10¹² cells per kilogram or from about 10¹² to about 10¹³ cells per kilogram or from about 10¹³ to about 10¹⁴ cells per kilogram of a recipient's body weight.

The pharmaceutical compositions may also contain additional therapeutic agents for treatment of the particular targeted disorder. For example, a pharmaceutical composition may also include cytokines and growth factors (interleukins, interferons, FGF, VEGF, PDGF, PIGF, STAT etc.). Such additional factors and/or agents may be included in the pharmaceutical composition to produce advantages of the therapeutic approaches disclosed herein, i.e., provide improved therapeutic efficacy with reduced systemic toxicity.

In other aspects, the invention provides a method for cell therapy, comprising administering the cell population described herein, or pharmaceutically acceptable composition thereof, to a human subject in need thereof. In various embodiments, the methods described herein are used to treat blood (malignant and non-malignant), bone marrow, immune diseases, and infectious diseases. In various embodiments, the human subject has a condition comprising one or more of lymphopenia, a cancer, an immune deficiency, an autoimmune disease. Examples of diseases include various autoimmune disorders, including but not limited to, alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barre syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjogren's syndrome, systemic lupus, erythematosus, some forms of thyroiditis, some forms of uveitis, vitiligo, granulomatosis with polyangiitis (Wegener's). Hematological malignancies that can be treated include but are not limited to acute and chronic leukemias, lymphomas, multiple myeloma and myelodysplastic syndromes. Infectious diseases that can be treated include but are not limited to HIV—(human immunodeficiency virus), RSV—(Respiratory Syncytial Virus), EBV—(Epstein-Barr virus), CMV—(cytomegalovirus), adenovirus- and BK polyomavirus-associated disorders. Other conditions include bone marrow failure syndrome, and certain genetic disorders (e.g., a genetic disorder impacting the immune system). In some embodiments, the subject has cancer, such as a solid tumor, including but not limited to, tumor of the brain, prostate, breast, lung, colon, uterus, skin, liver, bone, pancreas, ovary, testes, bladder, kidney, head, neck, stomach, cervix, rectum, larynx, or esophagus.

The cell populations can be formulated, dosed, and administered in a fashion consistent with good medical practice. Factors for consideration in this context include the particular disease or disorder being treated, the particular mammal being treated (e.g., human), the clinical condition of the individual patient, the cause of the disease or disorder, the site of delivery of the agent, the method of administration, the scheduling of administration, and other factors known to medical practitioners. The therapeutically effective amount of the cells to be administered will be governed by such considerations.

One may administer other compounds, such as cytotoxic agents, immunosuppressive agents and/or cytokines or growth factors (e.g., stem cell factor, thrombopoietin, transforming growth factor (TGF)-α or β, fibroblast growth factors (FGF), angiopoietin (Ang) family of growth factors, insulin-like growth factors, granulocyte-macrophage colony-stimulating factor, TNF-α or β, VEGF, interleukins (e.g., IL-2, 6, 7, 8 10, 12, 15 etc.,) and interferons (e.g., INF-alpha or gamma)) with the cells. The combined administration includes co-administration, using separate formulations or a single pharmaceutical formulation, and consecutive administration in either order, wherein preferably there is a time period while both (and all) active agents simultaneously exert their biological activities.

With respect to the use of HLA-edited B cells (e.g., derived from gene-edited iPSCs), the subject can be matched at retained HLA loci, such as one or more (or all) of HLA-B, HLA-C, and HLA-DRB1.

In certain embodiments, prior to therapy, patients may underdo lymphodepleting chemotherapy with a chemotherapeutic agent, such as fludarabine or cyclophosphamide (or other known process).

As used herein, the term “about” means±10% of the associated numerical value.

Certain aspects and embodiments of this disclosure are further described with reference to the following examples.

### EXAMPLES

### Example 1—ETV2 Over-Expression Increases the Yield of Hemogenic Endothelial Cells and Enhances the CD34+ Cell Formulation During iPSC Differentiation but does not Affect Pluripotency

### Methods

iPSCs were developed from hCD34+ cells by episomal reprogramming as known in the art and essentially as described in Yu, et al. Induced pluripotent stem cell lines derived from human somatic cells, Science 318, 1917-1920, (2007); and J. Yu, et al. Human induced pluripotent stem cells free of vector and transgene sequences. Science 324, 797-801, (2009). Embryoid Bodies and hemogenic endothelium differentiation was performed essentially as described in: R. Sugimura, et al., Haematopoietic stem and progenitor cells from human pluripotent stem cells. Nature 545, 432-438, (2017); C. M. Sturgeon, et al, Wnt signaling controls the specification of definitive and primitive hematopoiesis from human pluripotent stem cells. Nat Biotechnol 32, 554-561, (2014); J. Yu, et al. Induced pluripotent stem cell lines derived from human somatic cells. Science 318, 1917-1920, (2007); and J. Yu, et al. Human induced pluripotent stem cells free of vector and transgene sequences. Science 324, 797-801, (2009).

Briefly, hiPSC were dissociated and resuspended in media supplemented with L-glutamine, penicillin/streptomycin, ascorbic acid, human holo-Transferrin, monothioglycerol, BMP4, and Y-27632. Next, cells were seeded in 10 cm dishes (EZSPHERE or low attachment plate) for the EB formation. On Day 1, bFGF and BMP4 were added to the medium. On Day 2, the media was replaced with a media containing SB431542, CHIR99021, bFGF, and BMP4. On Day 4, the cell media was replaced with a media supplemented with VEGF and bFGF. On day 6, the cell media was replaced with a media supplemented with bFGF, VEGF, interleukin (IL)-6, IGF-1, IL-11, SCF, and EPO. Cells were maintained in a 5% CO₂, 5% O₂, and 95% humidity incubator. To harvest the CD34+ cells, the EBs were dissociated on day 8, cells were filtered through a 70 m strainer, and CD34+ cells were isolated by CD34 magnetic bead staining.

### Results

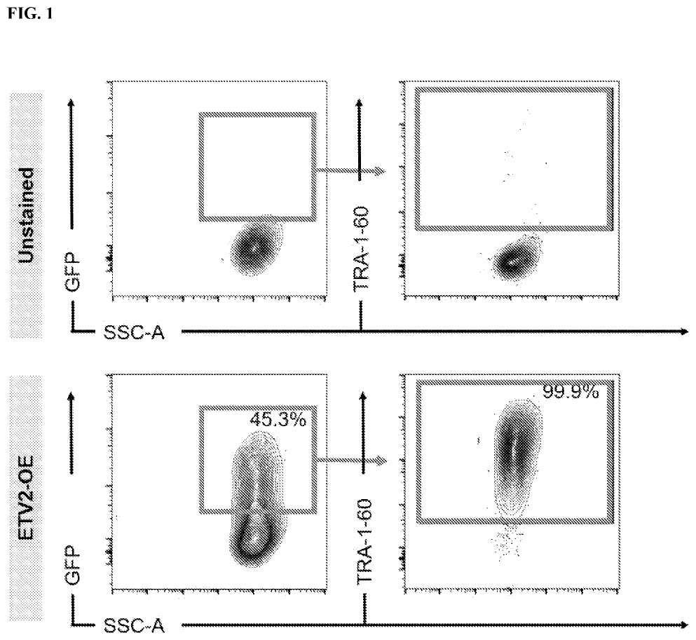

An adenoviral vector containing both ETV2 and GFP sequences under the control of the EF1A promoter was used to transduce induced pluripotent stem cells (iPSCs). After the transduction, about 45% of the iPSC culture was observed to be GFP positive, thus confirming ETV2 overexpression (ETV2-OE). It was further observed that ETV2-OE in iPSC cells preserves the pluripotency properties of iPSCs as shown by the stemness marker expression TRA-1-60 (FIG. 1). FIG. 1 shows FACS plots representative of transduction efficiency of iPSC with an adenoviral vector to overexpress the ETV2 and the GFP sequences.

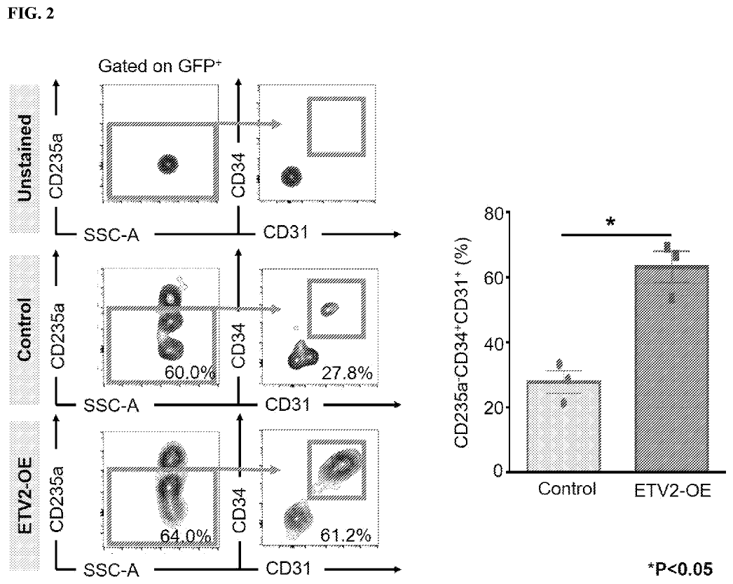

Next, the ETV2-OE-iPSCs were differentiated (along with control iPSCs transduced with a vector bearing the GFP sequence without ETV2) to embryoid bodies and subsequently to hemogenic endothelial cells (Strugeon et al., 2014). The results suggest that the overexpression of ETV2 boosts the formation of hemogenic endothelial cells as demonstrated by the expression of the CD34⁺ and CD31⁺ markers within the CD235a-population (FIG. 2). Specifically, FIG. 2 shows representative flow cytometric analysis of hemogenic endothelial cells (defined here as CD235a-CD34+CD31+) and relative quantification demonstrates that ETV2-OE enhances the formation of hemogenic endothelial cells as compared to controls.

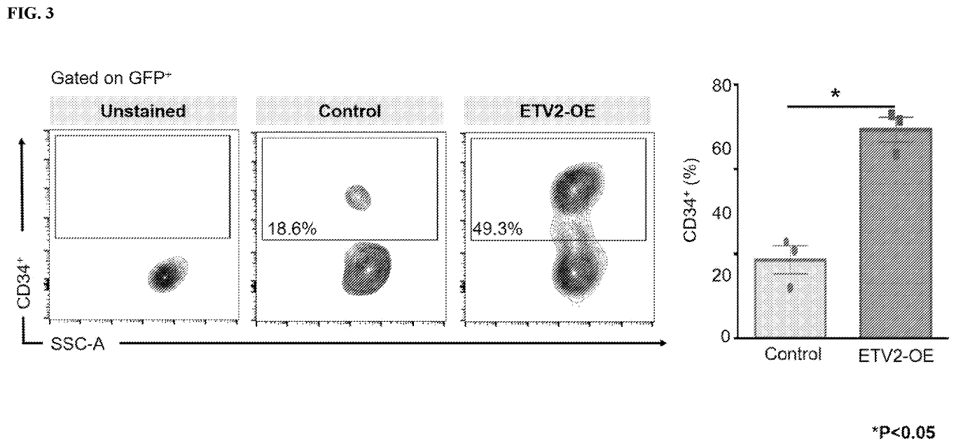

Moreover, the results suggest that ETV2-OE enhances the formation of the CD34⁺ cells (FIG. 3). FIG. 3 shows representative flow cytometric analysis of CD34+ cells and relative quantification demonstrates that ETV2-OE enhances the CD34+ cell formation.

Overall, these data indicate that ETV2 overexpression in iPSCs does not affect their pluripotency properties and facilitates their ability to undergo the hemogenic endothelial and hematopoietic differentiations.

### Example 2—iPSC-Derived HSCs Generated with Piezol Activation Undergo T Cell Differentiation Similar to Bone Marrow-Derived HSCs

### Methods

To analyze the EHT, EB-derived CD34+ cells were suspended in medium containing Y-27632, TPO, IL-3, SCF, IL-6, IL-11, IGF-1, VEGF, bFGF, BMP4, and FLT3. After the cells had adhered to the bottom of the wells for approximately 4-18 hours (by visual inspection), Yoda1 was added to the cultures. After 4-7 days, the cells were collected for analysis.

iPSCs were differentiated to embryoid bodies for 8 days. At day 8, CD34+ cells from iPSC-derived embryoid bodies were harvested and cultured for additional 5 to 7 days to induce endothelial-to-hematopoietic (EHT) transition. Then, CD34+ cells were harvested from the EHT culture between day 5 to day 7 for further hematopoietic lineage differentiation.

CD34+ cells, harvested from the EHT culture between day 5-7 (or total of day 13-21 differentiation from iPSCs), were seeded in 48-well plates pre-coated with rhDL4 and RetroNectin. T lineage differentiation was induced in media containing aMEM, FBS, ITS-G, 2BME, ascorbic acid-2-phosphate, Glutamax, rhSCF, rhTPO, rhIL7, FLT3L, rhSDF-1a, and SB203580.

Between day 2 to day 6, 80% of the media was changed every other day. At D7, cells were transferred into new coated plates and analyzed for the presence of pro-T cells (CD34+CD7+CD5+/−).

Between day 8 to day 13, 80% of the media was changed every other day. At D14, 100,000 cells/wells were transferred to a new coated plate and the cells analyzed for the presence of pre-T cells (CD34−CD7+CD5+/−).

Between day 15 to day 20, 80% of the media was changed every other day. Cells were harvested at D21, and the cells were analyzed for CD3, CD8, CD5, CD7, TCRab expression, as surrogates for T cells, via FACS, and/or activated using CD3/CD28 beads to evaluate their functional properties.

After 21 days of differentiation, cells were collected and re-seeded at approximately 80,000 cells into new 96-well culture plates in RPMI 1640 (no L-glutamine; no phenol red) plus FBS, L-glutamine, IL-2, and then activated with 1:1 CD3/CD28 beads. After 72 hours of activation with CD3/CD28 beads, cells were analyzed for CD3, CD69, CD25 expression by FACS and IFN-γ expression using RT-qPCR. The supernatant was analyzed by ELISA.

### Results

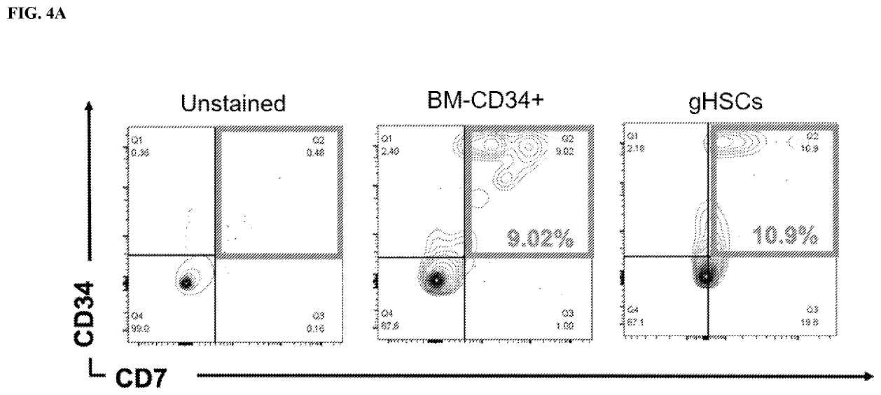

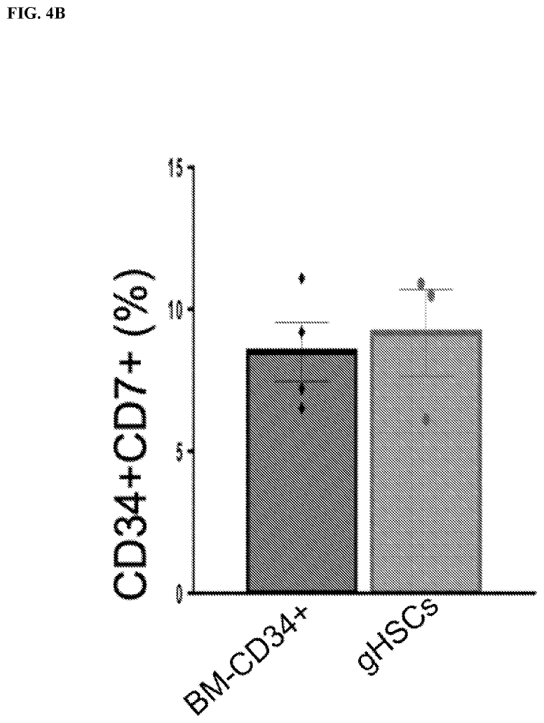

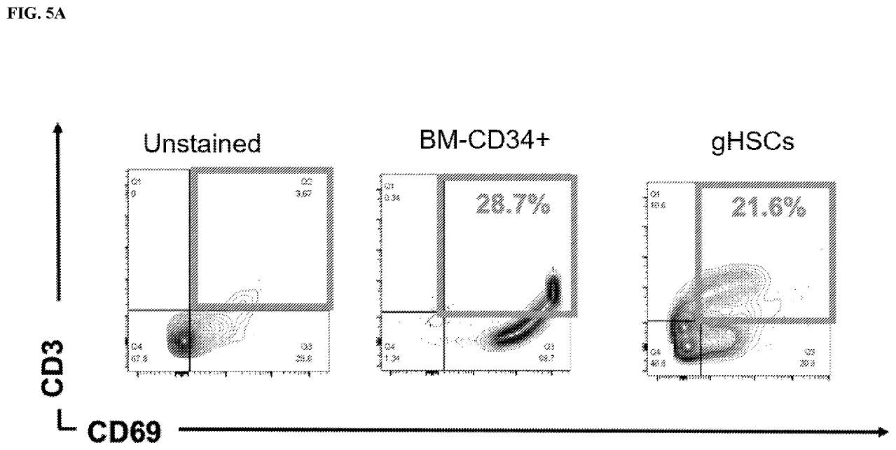

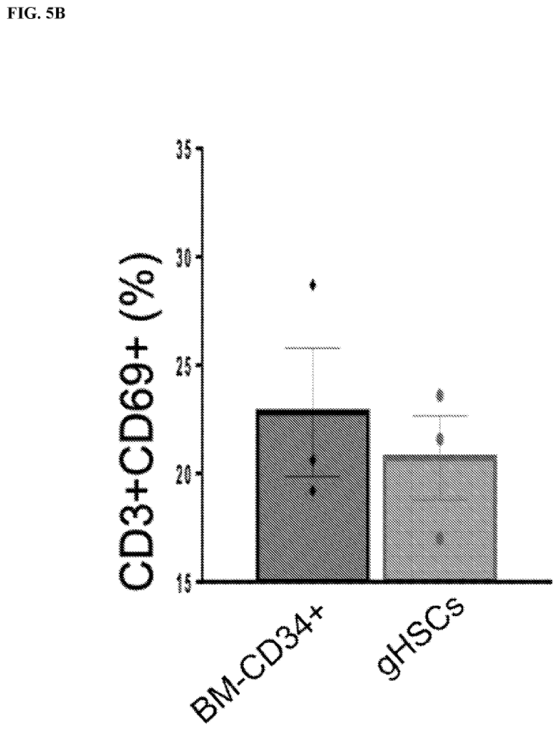

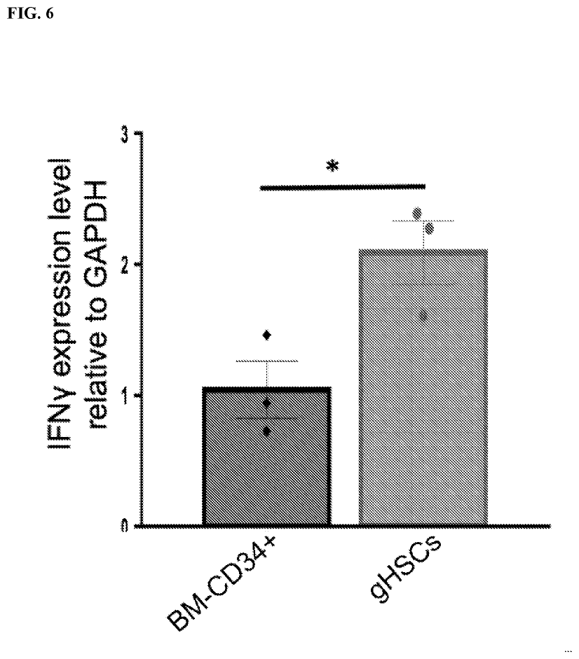

FIG. 4A and FIG. 4B show that iPSC-derived HSCs that are derived with EHT of D8 CD34+ cells (in this example with Piezol activation) undergo pro-T cell differentiation similar to bone marrow (BM)-HSCs. Further, FIG. 5A and FIB. 5B show that iPSC-derived HSCs generated with EHT of D8 CD34+ cells (in this example with Piezol activation) undergo T cell differentiation and can be activated with CD3/CD28 beads similar to BM-HSCs. FIG. 6 shows that iPSC-derived HSCs generated with EHT of D8 CD34+ cells (in this example with Piezol activation) can differentiate to functional T cells, as demonstrated by INFγ expression upon stimulation with CD3/CD28 beads. Together, these results demonstrate that EHT of D8 CD34+ cells enhances the ability to further differentiate to hematopoietic lineages ex vivo.

### Example 3—Evaluating Off-Target Editing in HLA Knockout HSCs

HLA typing of the HLA edited (e.g., triple knock out) HSC clones was performed to check for unwanted editing and to ensure that no major editing events, e.g., deletion(s), occurred within other regions of chromosome 6. Sequencing methods and analyses were performed to evaluate the degree of gRNA off-target activity and to select gRNAs that represent a low risk of affecting non-target HLA genes.

Sequencing was performed by using in situ break labelling in fixed and permeabilized cells by ligating a full-length P5 sequencing adapter to end-prepared DSBs. Genomic DNA was extracted, fragmented, end-prepared, and ligated using a chemically modified half-functional P7 adapter. The resulting DNA libraries contained a mixture of functional DSB-labelled fragments (P5:P7) and non-functional genomic DNA fragments (P7:P7). Subsequent DNA sequencing of the DNA libraries enriched for DNA-labelled fragments, eliminating all extraneous, non-functional DNA. As the library preparation is PCR-free, each sequencing read obtained was equivalent to a single labelled DSB-end from a cell. This generated a DNA break readout, enabling the direct detection and quantification of genomic DSBs by sequencing without the need for error-correction and enabled mapping a clear list of off-target mutations.

Table 1 below summarizes the results of the editing strategy in two representative clones relative to a wild-type cell.

TABLE 1
Clonal HSC HLA knockouts.

| Sample ID | Locus | Allele 1 | Allele 2 | Comments |
|---|---|---|---|---|
| WT (gHSC) | A | A*01:01:01 | A*01:01:01 | Not affected |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | DPB1*01:01:01 | DPB1*04:01:01 | Not affected |
| | DQA1 | DQA1*05:01:01 | DQA1*05:01:01 | Not affected |
| | DQB1 | DQB1*02:01:01 | DQB1*02:01:01 | Not affected |
| | DRB1 | DRB1*03:01:01 | DRB1*03:01:01 | Not affected |
| | DRB345 | DRB3*01:01:02 | DRB3*01:01:02 | Not affected |
| Clone #1 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #2 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |

Table 2 provides a non-limiting example of gRNAs used in the experiments which can be used to knock out expression of indicated HLA genes and haplotypes.

TABLE 2
Exemplary gRNA sequences

| gRNA ID | Spacer sequence |
|---|---|
| HLA-A | CGTAGCCCACGGCGATGAAG (SEQ ID NO: 1) |
| | ATTTCTTCACATCCGTGTCC (SEQ ID NO: 2) |
| | GTCTCCTGGTCCCAATACTC (SEQ ID NO: 3) |
| | GTAGCCCACGGCGATGAAGC (SEQ ID NO: 4) |
| | TAGCCCACGGCGATGAAGCG (SEQ ID NO: 5) |
| | GAGGGTTCGGGGCGCCATGA (SEQ ID NO: 6) |
| | CCGGAACACACGGAATGTGA (SEQ ID NO: 7) |
| | ACAGCGACGCCGCGAGCCAG (SEQ ID NO: 8) |
| | CCACTCACAGATTGACCGAG (SEQ ID NO: 9) |
| | CGACGCCGCGAGCCAGAGGA (SEQ ID NO: 10) |
| | CAGATTGACCGAGTGGACCT (SEQ ID NO: 11) |
| | CAGACTGACCGAGTGGACCT (SEQ ID NO: 12) |
| | ACAGACTGACCGAGTGGACC (SEQ ID NO: 13) |
| | GCCGGGCCGGGACACGGATG (SEQ ID NO: 14) |
| | CCAGGAGACACGGAATGTGA (SEQ ID NO: 15) |
| | CCAGTCACAGACTGACCGAG (SEQ ID NO: 16) |
| | TCGACAGCGACGCCGCGAGC (SEQ ID NO: 17) |
| HLA- | GAGATACATCTACAACCGGG (SEQ ID NO: 18) |
| DPB1 | TGGAGAGATACATCTACAAC (SEQ ID NO: 19) |
| | GGTTGTAGATGTATCTCTCC (SEQ ID NO: 20) |
| | GGAGAGATACATCTACAACC (SEQ ID NO: 21) |
| | AGGAATGCTACGCGTTTAAT (SEQ ID NO: 22) |
| HLA- | CAGATACATCTATAACCGAG (SEQ ID NO: 23) |
| DQB1 | CGAGTACTGGAACAGCCAGA (SEQ ID NO: 24) |
| | CCCGTTGGTGAAGTAGCACA (SEQ ID NO: 25) |
| | GTGCTACTTCACCAACGGGA (SEQ ID NO: 26) |
| | AGGTCGTGCGGAGCTCCAAC (SEQ ID NO: 27) |
| | AGGTGACAGTTGCTACCCGA (SEQ ID NO: 28) |
| | TCCCGTTGGTGAAGTAGCAC (SEQ ID NO: 29) |
| | AACTACGAGGTGGCGTACCG (SEQ ID NO: 30) |
| | CTGTTCCAGTACTCGGCAAC (SEQ ID NO: 31) |
| | ACTACGAGGTGGCGTACCGC (SEQ ID NO: 32) |
| | CGAAGCGCACGTACTCCTCT (SEQ ID NO: 33) |

The results show that the editing strategy was successful in selectively targeting the HLA-A, DPB1, and DQB1 genes without affecting the other HLA genes or introducing major deletion elsewhere.

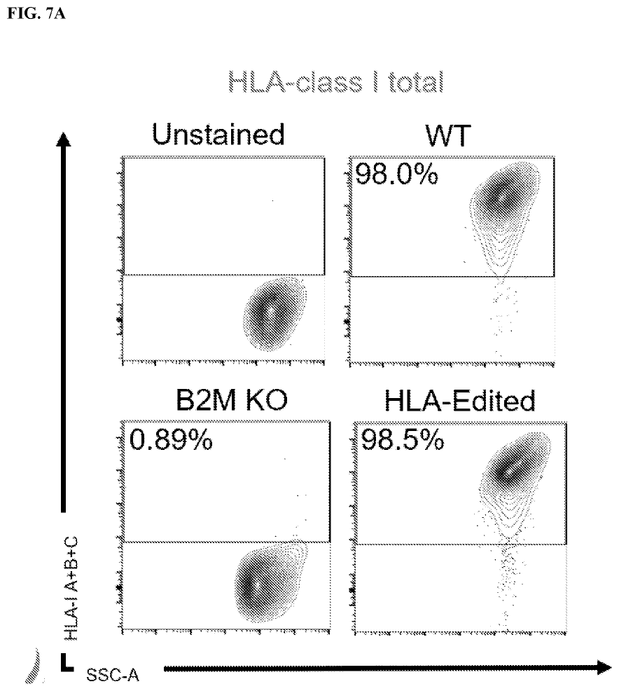

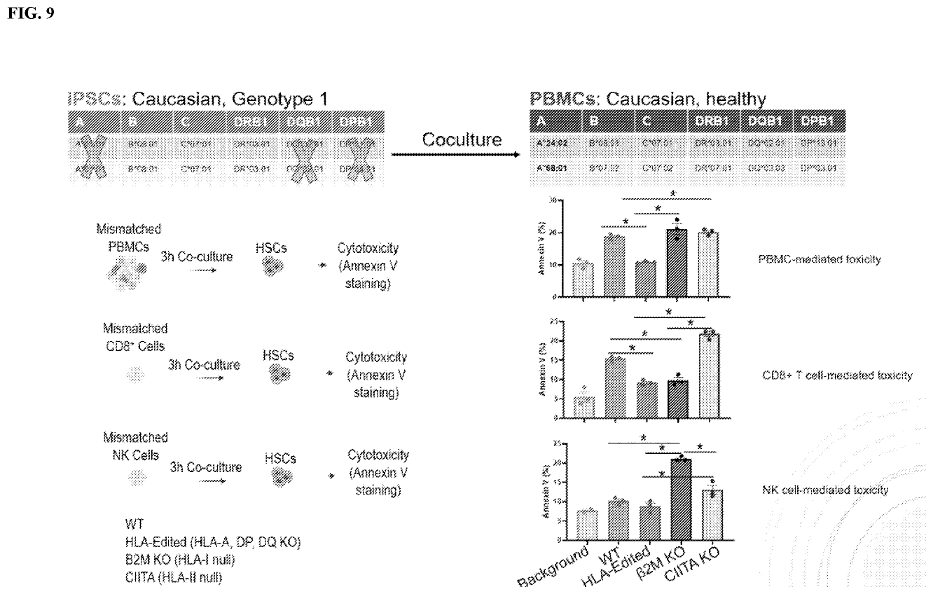

These results were confirmed by a phenotypic analysis of the HLA edited clones by FACS and immunofluorescence. As shown in FIGS. 9A and B, HLA edited cells tested positive for overall expression of HLA class-I molecules, comparable to the overall expression of HLA class-I molecules of wild-type cells (gHSCs). Specific expression of HLA-A via immunofluorescence confirmed that HLA-A was not expressed in the HLA edited cells, corroborating the finding that the gene editing strategy was successful in deleting only the HLA-A gene. Specifically, FIG. 7A, shows that the HLA edited cells were all positive for class-I like HLA to the same extent as the wild type (WT) (i.e., non-HLA-edited) cells. This result indicates that despite the deletion of HLA-A, other class-I molecules like HLA-B and C were expressed and not affected by the gene editing strategy.

To confirm that the HLA-A gene was deleted, specific expression of the HLA-A was analyzed with immunofluorescence. As can be seen in FIG. 7B, HLA-A was not expressed in the HLA edited clone indicating that the gene editing strategy was efficient in specifically deleting the HLA-A gene only. Such preservation of overall class-I expression with deletion of HLA-A will facilitate patient matching while avoiding NK-cell mediated rejection.

### Example 4—Evaluating Pluripotency and Immunocompatibility of HLA Edited HSCs

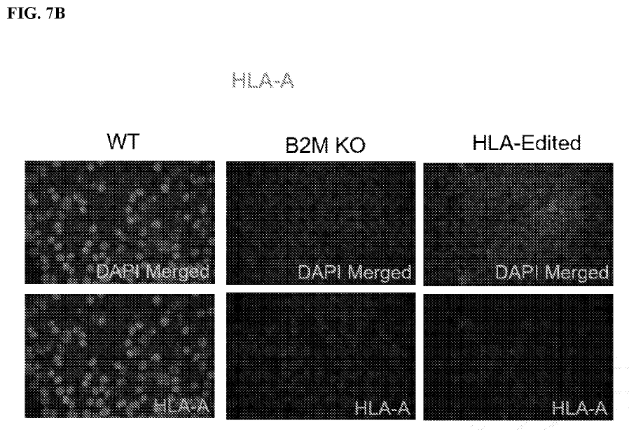

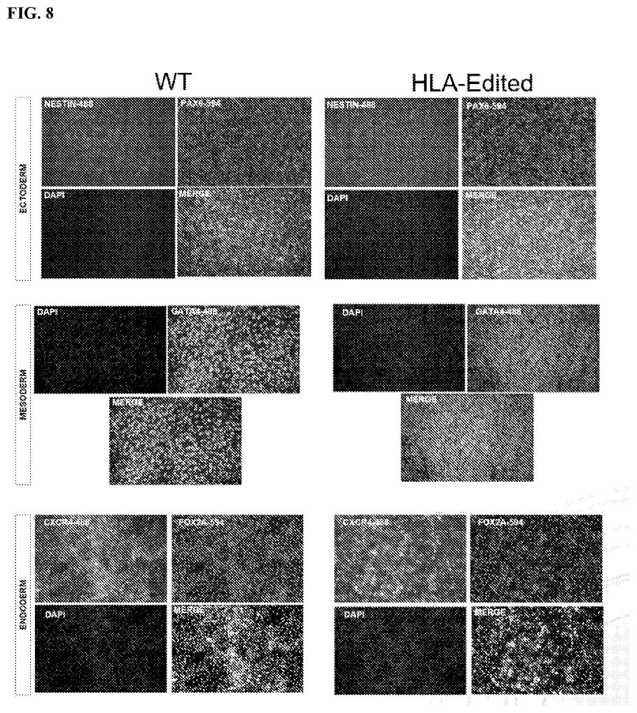

The ability of HLA edited cells to preserve pluripotency was evaluated. As shown in FIG. 8, immunofluorescence evaluation of the HLA edited iPSC clones indicated that they maintained trilineage differentiation, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

HLA class I molecules are expressed on the surface of all nucleated cells and if the HLA class I molecules are mismatched between donor and recipient, then the cells could be recognized and killed by CD8+ T cells. Additionally, HLA mismatching could lead to cytokine release syndrome (CRS) and graft-versus-host disease (GVHD). Conversely, the complete deletion of HLA-I molecules, via B2M KO, would make the cell a target of NK cell-mediated cytotoxicity. The preservation of overall class-I expression with deletion of HLA-A can facilitate patient matching while preventing the NK-cell mediated rejection. Thus, the immunocompatibility of the HLA edited HSCs was tested by co-culture with peripheral blood mononuclear cells (PBMCs) to evaluate if the immune cells would reject a graft of the HLA edited and wild type HSCs (gHSCs).

Wild-type (i.e., gHSCs) and HLA edited HSCs were co-cultured with PBMCs matching the HLA-B and HLA-C markers, but with mismatched HLA-A. B2M KO HSCs lacking expression of HLA class-I molecules and CIITA KO HSCs lacking expression of class-II molecules were used as controls to compare the degree of PBMC-mediated cytotoxicity for HLA-null and mismatched HLA, respectively. FIG. 9 shows the results of the PBMC-mediated cytotoxicity assay in the co-cultures as measured by an annexin V staining. The results show that deletion of HLA-A in the HLA edited HSCs protects the cells from PBMC-mediated cytotoxicity, while WT, B2M KO, and CIITA KO were susceptible to PBMC-mediated cytotoxicity. Co-cultured HSCs with sorted CD8+ T cells from the same PBMC donor protected HLA edited and B2M KO HSCs from CD8+ T cell cytotoxicity. Conversely, co-cultured HSCs with sorted NK cells only protected the WT and HLA edited cells from the NK cell-mediated cytotoxicity.

In summary, the immune compatibility results show that the CD8+ T cells present in the PBMC samples were responsible for killing the cells with mismatched HLA molecules (WT and CIITA KO), while the NK cells present in the PBMCs were responsible for killing the HLA-null cells (B2M KO). However, HLA edited HSCs are protected from CD8+ T cell-mediated cytotoxicity (because the mismatched HLA-A had been knocked out), and protected from NK cell-mediated cytotoxicity (because HLA class I molecule expression was largely preserved).

### Example 5—Evaluating the In Vivo Engraftment Potential of HLA Edited HSCs

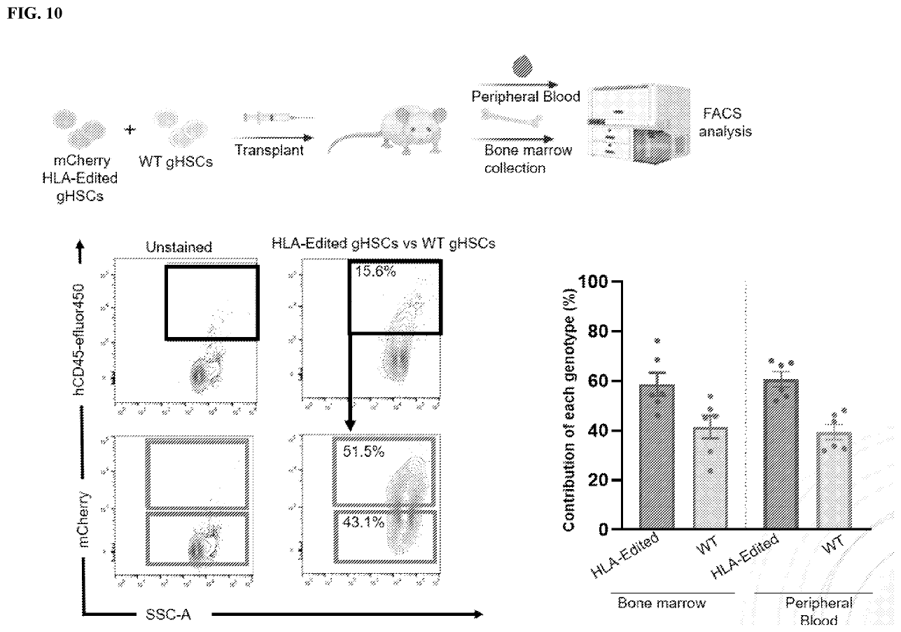

To evaluate the engrafting potential of HLA edited HSCs, the cells' ability to engraft in vivo was evaluated by a competitive transplant against WT HSCs. Equal proportions of mCherry HLA edited HSCs and wild-type HSCs (gHSCs) were admixed and transplanted into mice, where bone marrow (BM) and peripheral blood samples were recovered and evaluated by FACS to compare the relative amounts of each cell type present in the samples. As shown in FIG. 10, both the HLA edited HSCs and the WT HSCs contributed to approximately equal engraftment in the BM and peripheral blood samples. These results confirm that HLA edited HSCs (prepared according to this disclosure) are comparable to WT HSCs in their engraftment and reconstitution potential. Hence, it is expected that properties of the WT (unedited, parent) HSCs are consistent with that of the HLA edited HSCs of the present disclosure, for generating T cell lineages.

### Example 6—Differentiation of HLA Edited HSCs to CD4+/CD8+ T Cells

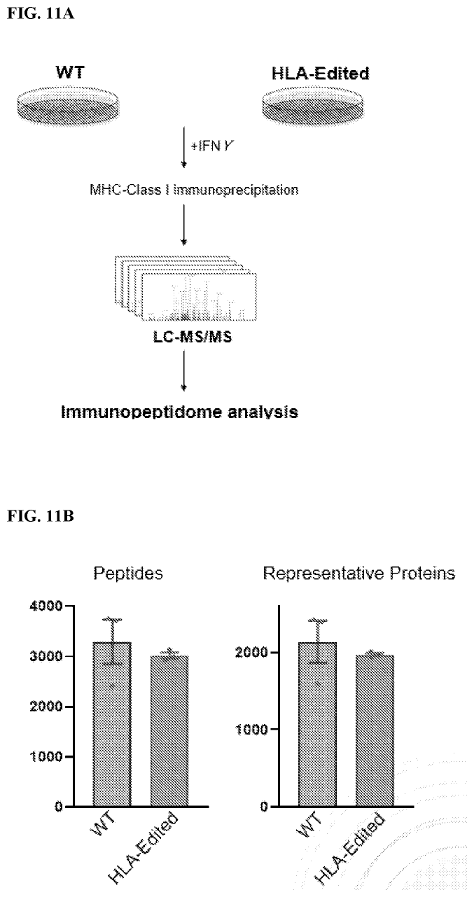

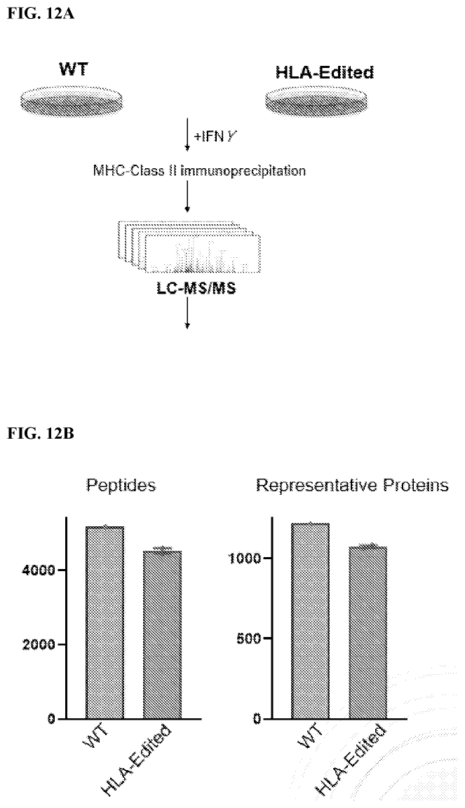

Antigen presenting cells (APCs) present antigens to helper CD4+ T cells through the HLA-II molecules. Activation of helper CD4+ T cells promotes the generation of antigen-specific CD8+ T cells which further develop into antigen-specific CTLs. Likewise, HLA Class I molecules are expressed on the surface of all nucleated cells and display peptide fragments of proteins from within the cell to CD8+ CTLs. CTLs induce cytotoxic killing of target (infected) cells upon recognition of HLA-I-peptide complex expressed on the cell surface. Hence, a study was carried out to determine if deletion of HLA-A impacts the edited HSCs' class I peptide presentation. As shown in FIGS. 11A and B, immunopeptidome analysis shows that the deletion of HLA-A does not impact overall class I peptide presentation. HLA-A edited cells showed comparable peptide and protein presentation when compared to wild type HSCs (gHSCs). Further, as shown in FIGS. 12A and B, deletion of HLA-DQB1 and HLA-DPB1 does not impact overall class II peptide presentation by macrophages differentiated from the HSCs. Together, these data suggest that despite the deletion of HLA-A, HLA-DQ, and HLA-DP molecules, the cells (and their derived lineages) preserve their ability to present a broad spectrum of class I and II peptides.

### Example 7—In Vivo Testing of Antigen-Mediated Immune Response

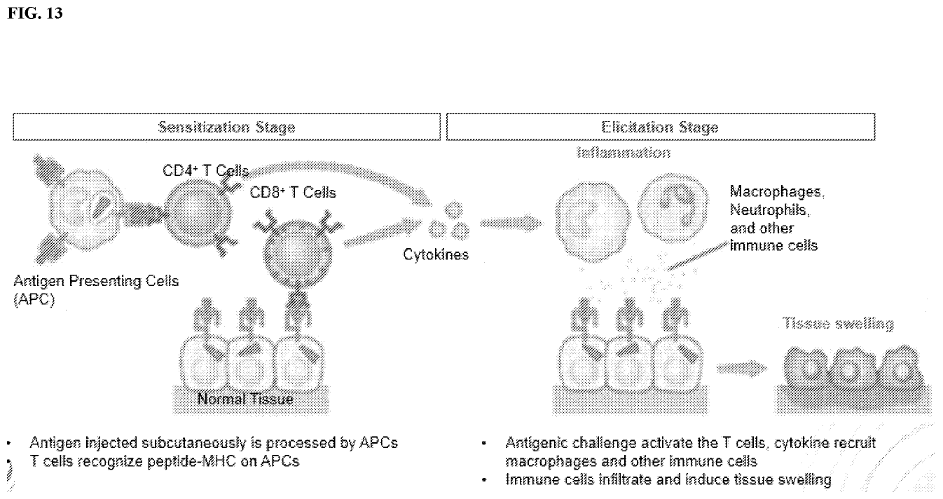

FIG. 13 is a schematic illustration of a Delayed Type Hypersensitivity Reaction, showing the sensitizing and eliciting stages of an antigen presentation. Briefly, upon antigen injection, antigen is processed by antigen presenting cells (APC) and presented by MHC Class II molecules on the APC surface. CD4+ T cells recognize peptide-MHC on antigen presenting cells (APCs). Upon antigenic challenge CD4+ helper T cells are activated and cytokines recruit macrophages and other immune cells, which induce tissue swelling.

A delayed-type hypersensitivity assay was performed on transplanted mice. Specifically, the mice were sensitized by subcutaneous injection of sheep Red blood cells as antigen. If the mice have a functional immune system, the APCs process the antigen and present peptide antigens to CD4+ T cells. Next, the mice were challenged by subcutaneous injection of the same antigen in the left paw. At this point the T cells are activated and secrete cytokines which recruit macrophages and other immune cells at the site of antigen injection creating tissue swelling. In this assay, a functional immune system resulted in the swelling of the left paw as measured with a micro caliper.

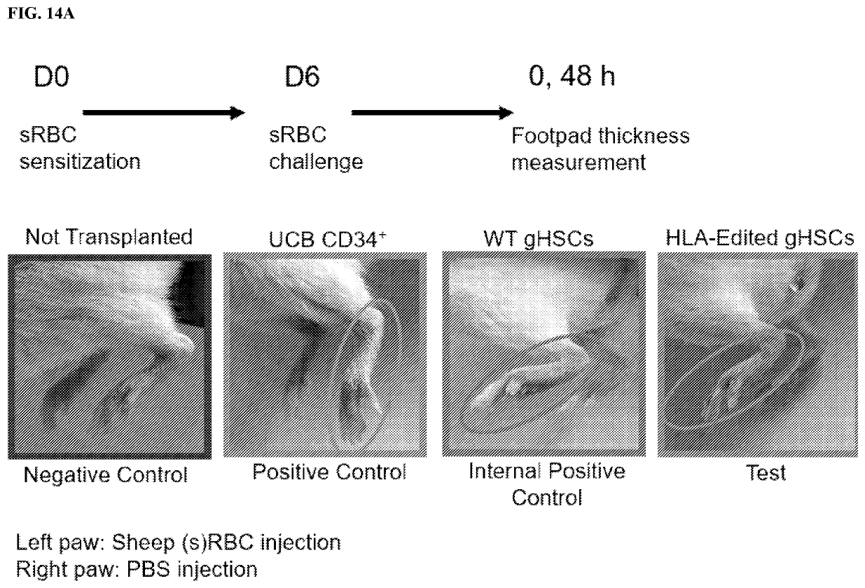

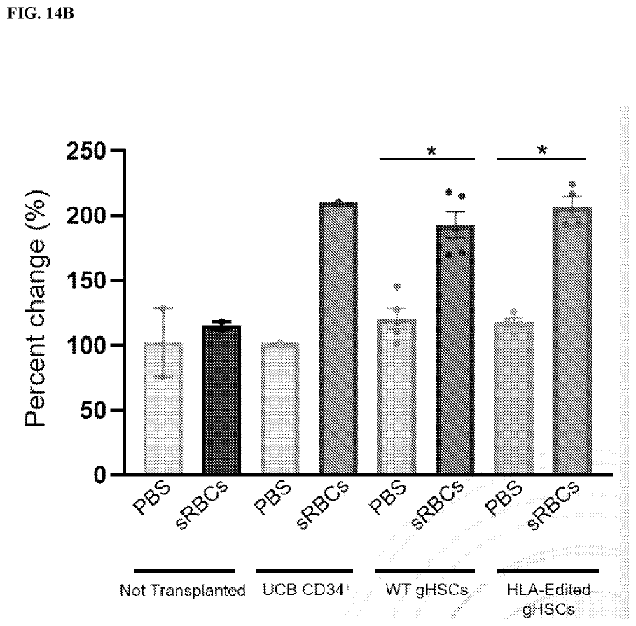

As can be seen in FIGS. 14A and B, the control (non-transplanted) mice did not show any left paw swelling as they are immunodeficient. Conversely, the mice transplanted with Cord Blood CD34+ cells showed tissue swelling and doubled the diameter of their left paw. A similar immune system response was found in both the mice transplanted with the WT (non-edited HSCs) and the HLA-edited HSCs.

### Example 8 Evaluating HSC-Derived T Cell (Pro-T Cell) Differentiation and Maturation

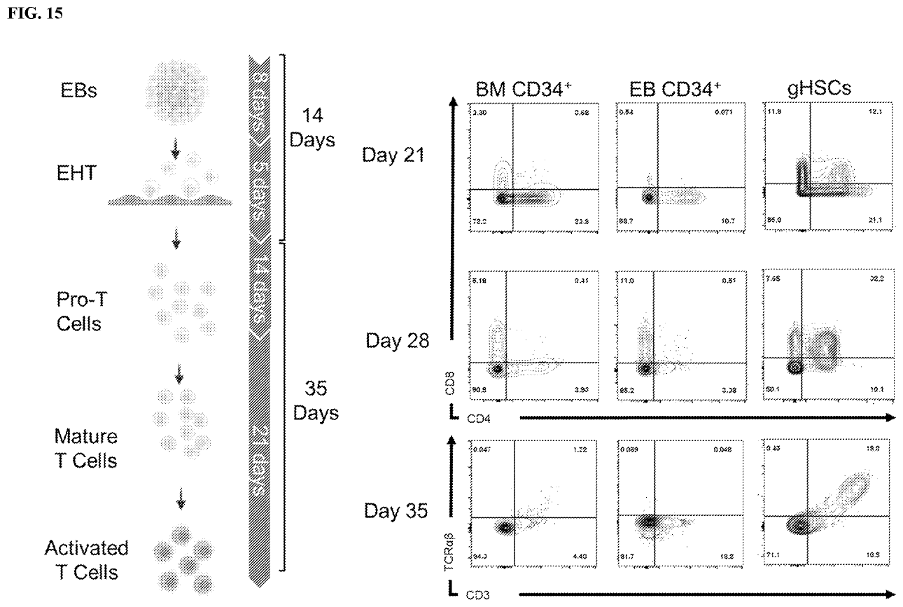

Next, the ability of the HSC-derived T cells (pro-T cells) to differentiate into mature T cells was tested. After a 35-day differentiation period, pro-T cells were evaluated by cell sorting for the presence of CD4+, CD8+, and AB+ T cell populations. As shown in FIG. 15, pro-T cells differentiated into CD4+, CD8+, and αβ+ T cells more efficiently than bone marrow (BM)-derived CD34+ cells and the CD34+ cells derived from the embryonic bodies (EB).

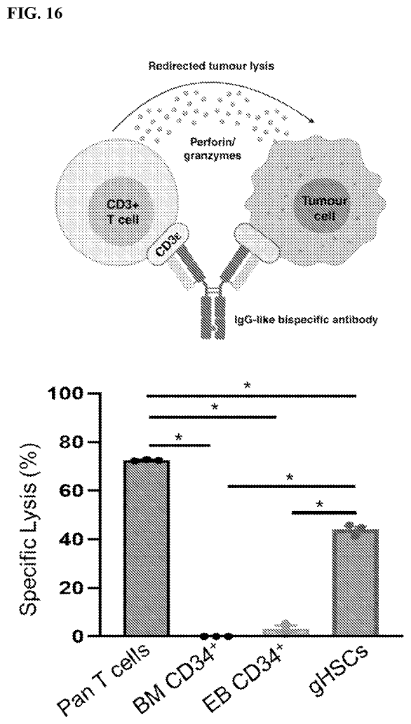

Next, to test the functional properties, each of the T cell populations were co-cultured with a CD19+ lymphoma cell line and an anti-CD3/CD-19 bispecific antibody. In this experimental model, the bispecific antibody engaged both the CD3 receptor on T cells and the CD19 cell surface receptor of the lymphoma cells, thus triggering T cell activation. The degree of activation was evaluated by measuring the subsequent T-cell mediated cytotoxicity in comparison to a Pan T cell control. As shown in FIG. 16, the pro-T cells exhibited a statistically significant outperformance in cytotoxicity in comparison to both the BM CD34+ T cells and the EB CD34+ T cells.

### Example 9—Evaluating HSC Properties of Developing into Pro-T Cells

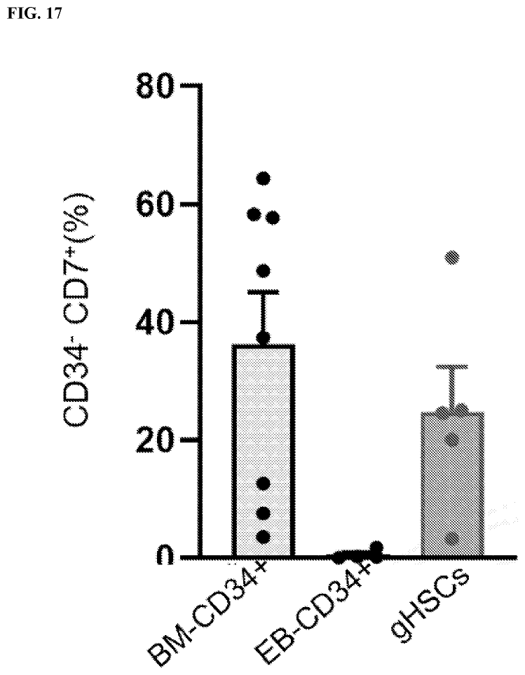

The ability of the HSCs to develop into pro-T cells was assessed by measuring the CD34-CD7+ markers on the pro-T cells. As shown in FIG. 17, FACS analysis showed that HSCs produced according to this disclosure successfully differentiated into CD34-CD7+ pro-T cells, as compared to bone marrow derived CD34+ cells or EB-derived CD34+ cells.

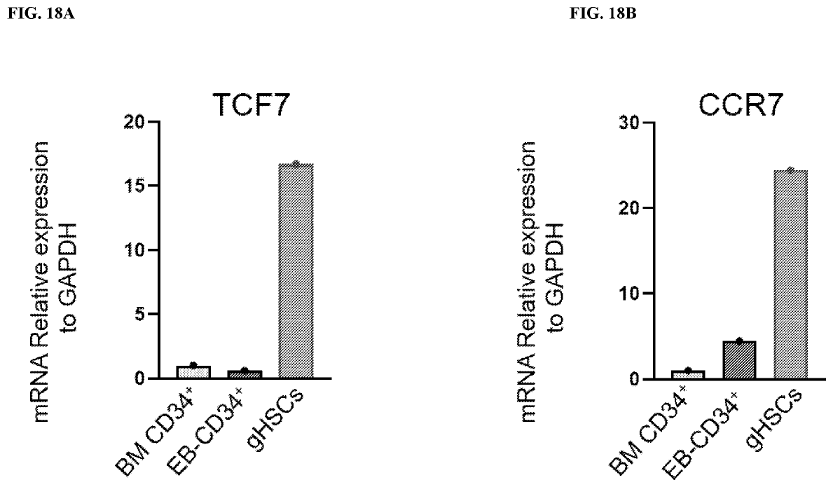

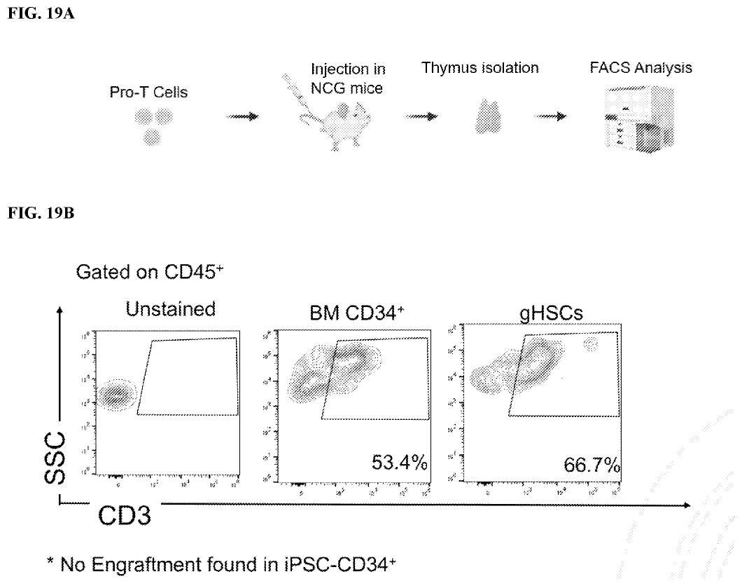

Next, the expression of T cell-specific transcription factors and thymus engrafting molecules were measured. FIG. 18A shows increased TCF7 expression and FIG. 18B shows increased CCR7 expression in the HSC-derived pro-T cells of the disclosure. FIG. 19A shows HSCs-derived Pro-T Cells engraft and differentiate in thymus. FIG. 19B shows FACS analysis of CD3 cell population of cells gated on a CD45+ cell population, which shows the superior engraftment and differentiation potential of the HSCs-derived Pro-T Cells in the thymus. Pro-T Cells of this example were prepared from HSCs using Piezol activation as already described.

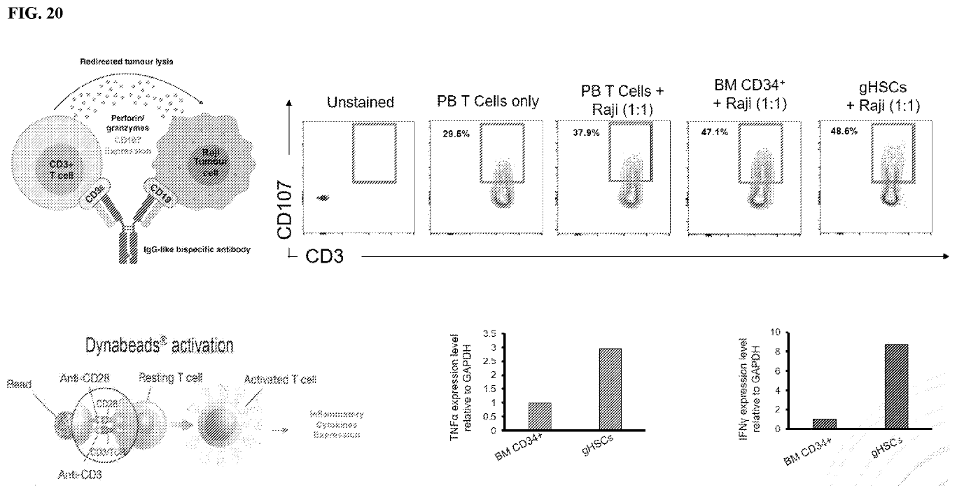

An in-vitro activation of the HSC-derived T cells were also measured, as illustrated in FIG. 20. Top panel of FIG. 20 shows FACS analysis of activated T cells from different sources, including the HSCs of the present disclosure (e.g., prepared using Piezol activation). T cells prepared from HSCs of the present disclosure demonstrated comparable or superior activation as measured by increased CD107 expression. The lower panel shows Dynabeads activation, where activated T cells express inflammatory cytokines. HSC-derived T cells according to the present disclosure (e.g., prepared using Piezol activation) expressed higher levels of inflammatory cytokines as exemplified by TNF-alpha and interferon gamma expression levels.

### Example 10: Differentiation of HLA Edited HSCs to Hematopoietic Lineages, Pro-Monocyte Macrophage Cells

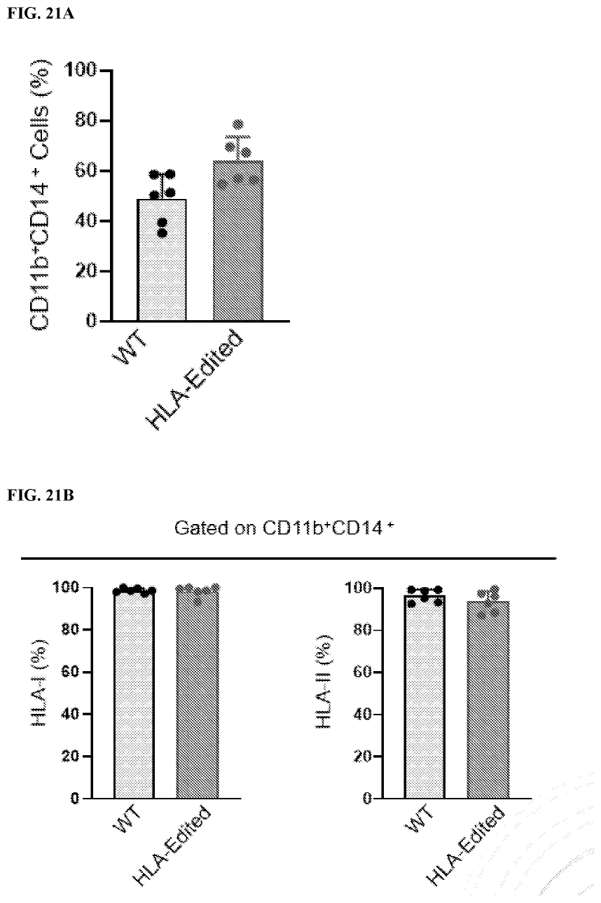

Experiments were carried out to determine if HLA deletion impacts the HSCs ability to differentiate into different types of immune cells. Using the process essentially as described in Example 2, HLA edited HSCs were differentiated to pro-Monocyte/Macrophage cells. It was determined that the HLA-edited HSCs were able to differentiate into monocyte/macrophage lineage comparable to WT (non-HLA-edited) HSCs as measured by their CD11b+-CD14+ expressions (FIG. 21A). Further, the CD11b+-CD14+ gated population showed equivalent HLA-I and HLA-II expression (FIG. 21B) indicating that HLA-edited HSCs also preserve the overall expression of both class I and class II molecules.

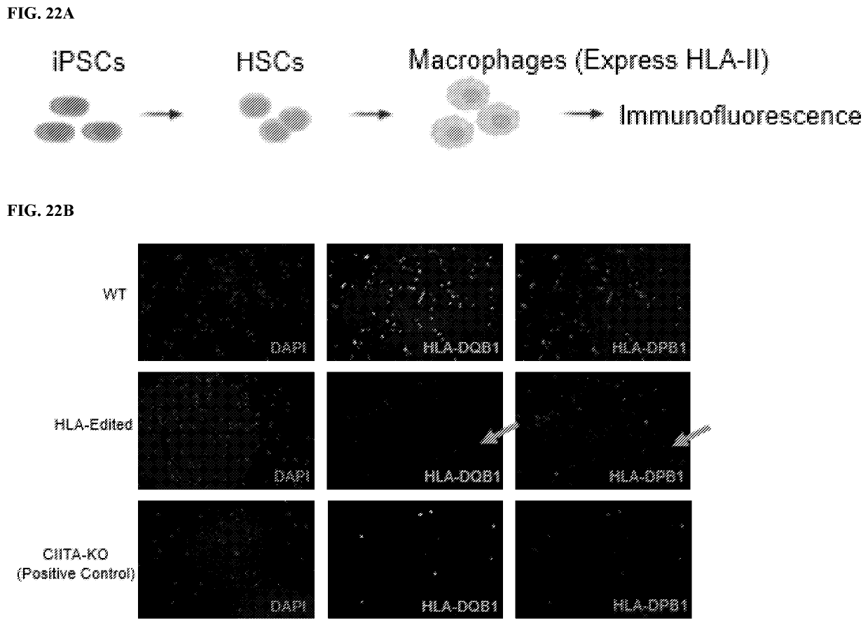

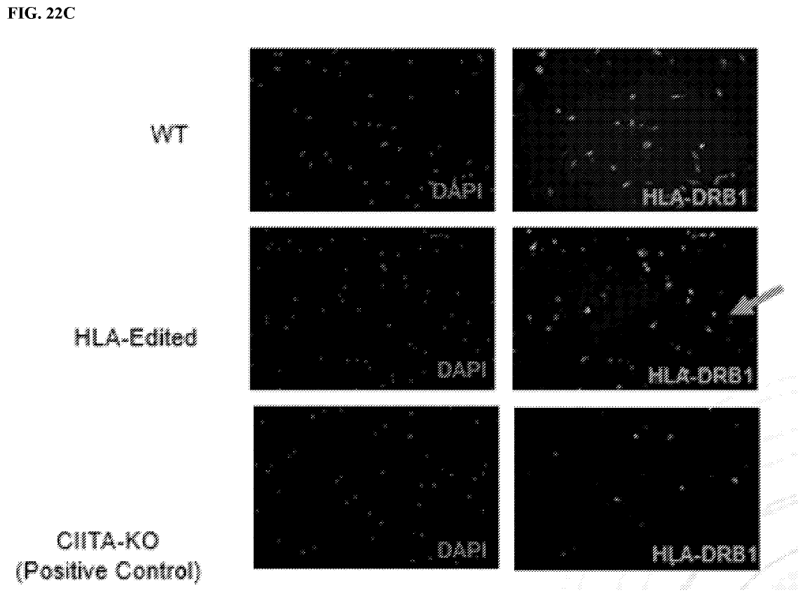

The overall expression of the other class-II molecules in HLA-DQB1 and HLA-DPB1 supported by the edited HSCs was evaluated, by evaluating the expression in macrophages differentiated from the HSCs. The design of the study is schematically shown in FIG. 22A. It was found that the deletion of HLA-DQB1 and HLA-DPB1 did not affect the expression of other HLA Class II molecules (FIG. 22B). For example, HLA-DR is comparably expressed in both WT and HLA-edited cells (FIG. 22C). In FIGS. 22B and 22C, CIITA-KO is as a positive control.

### Example 11: Differentiation of HLA Edited HSCs to Pro-Platelets

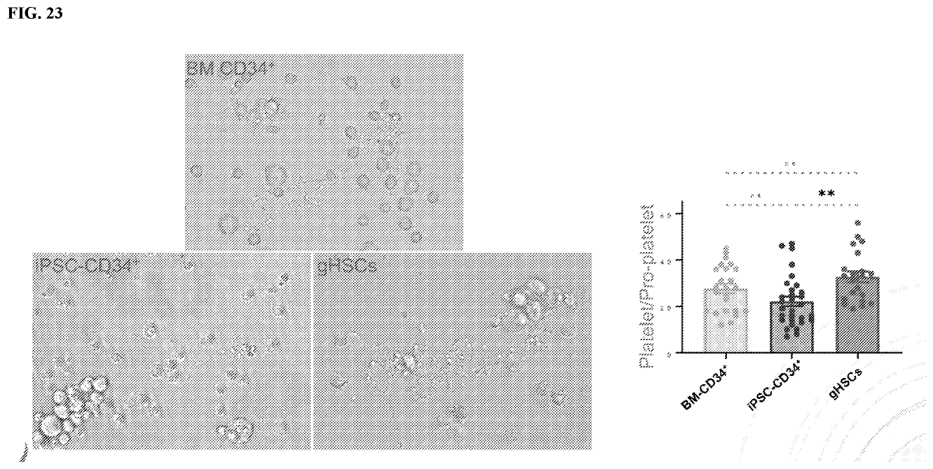

It was determined that the HLA-edited HSCs were able to differentiate into megakaryocytes (MK) and further into platelets. Differentiation was compared against bone marrow (BM)-derived CD34+ cells and iPSC-CD34+ cells. As shown in FIG. 23, the HLA-edited HSCs exhibited a statistically significant increase in platelet content compared to the BM CD34+ and iPSC-34+ cell populations. Thus, HLA-edited HSCs can differentiate into megakaryocytes (MK) which can further support differentiation into platelets.

### Example 12—Evaluating the Degranulation and Cytotoxicity of Immunocompatible HSC-Derived NK Cells

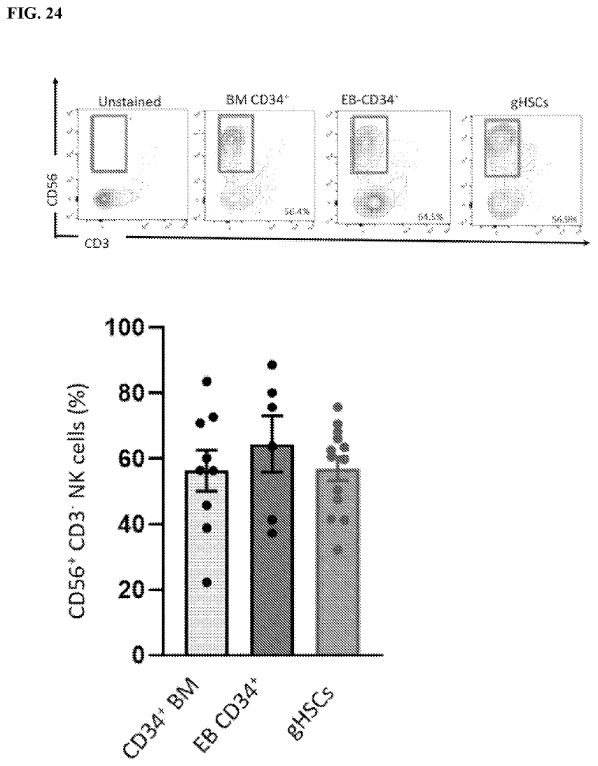

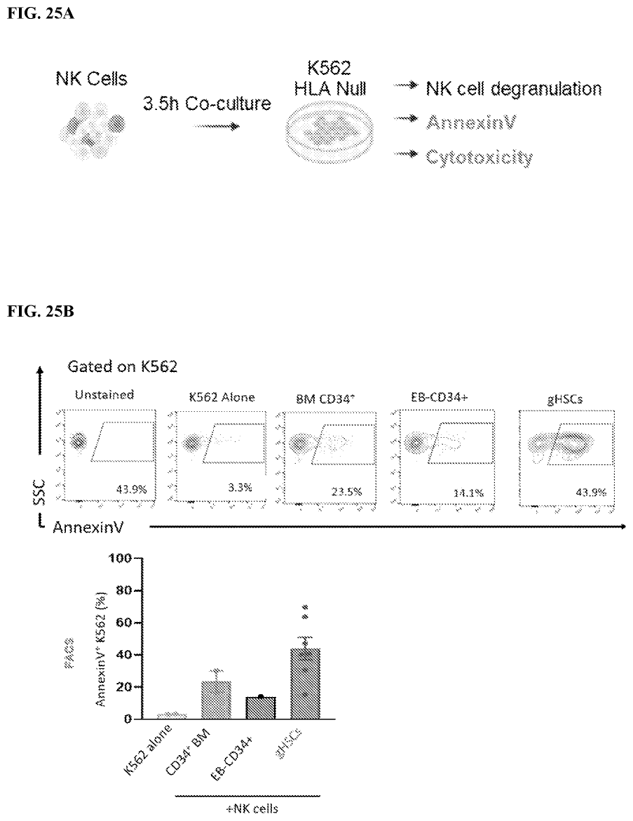

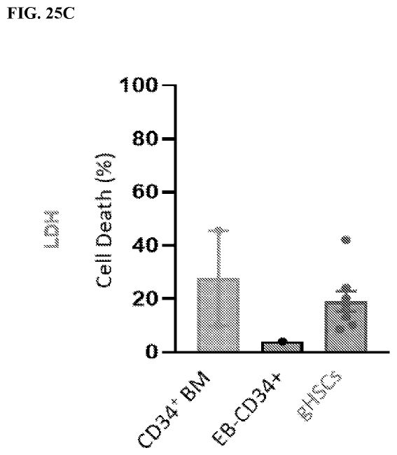

Next, triple knockout HSCs were evaluated for their ability to differentiate into NK cells which maintained their degranulation and cytotoxicity capabilities. As shown in FIG. 24, HSCs effectively differentiated into NK cells as determined by fluorescence-activated cell sorting (FACS) experiments, gated based on expression of the known NK cell surface marker, CD56. The HSCs demonstrated the ability to differentiate at least as well as CD34+BM and iPSC-EB CD34+ cell populations. To measure the ability of the HSC-derived NK cells to effectively kill tumor cells, an experimental protocol as shown in FIG. 25A was performed. The HSC-derived NK cells were co-cultured for 3.5 hours with K562 HLA-null cells, which are a human erythromyeloblastoid leukemia cell line derived from pleural effusion of a chronic myeloid leukemia patient. These cells expressed ligands for aNKR, and their lack of HLA cell-surface expression also contributes to NK cell activation by abrogating negative signaling through iNKRs. Therefore, these HLA-null cell lines have the potential to induce different functional profiles in NK cells and their subsets. After co-culturing, the degree of NK cell degranulation is measured using FACS and AnnexinV staining, as well as a cytotoxicity assay. As shown in FIG. 25B, AnnexinV staining demonstrated that HSC-derived NK cells exhibited a greater degree of activation from the HLA-null K562 cells than did the CD34+BM and iPSC-EB CD34+ cells. This was confirmed by the cytotoxicity assay results as shown in FIG. 25C.

### REFERENCES

1. Nianias, A. & Themeli, M. Induced Pluripotent Stem Cell (iPSC)-Derived Lymphocytes for Adoptive Cell Immunotherapy: Recent Advances and Challenges. Curr Hematol Malig Rep 14, 261-268 (2019).
2. Brauer, P. M., Singh, J., Xhiku, S. & Znhiga-Pflicker, J. C. T Cell Genesis: In Vitro Veritas Est?Trends Immunol 37, 889-901 (2016).
3. Kennedy, M. et al. T Lymphocyte Potential Marks the Emergence of Definitive Hematopoietic Progenitors in Human Pluripotent Stem Cell Differentiation Cultures. Cell Reports 2, 1722-1735 (2012).
4. Sturgeon, C. M., Ditadi, A., Awong, G., Kennedy, M. & Keller, G. Wnt Signaling Controls the Specification of Definitive and Primitive Hematopoiesis From Human Pluripotent Stem Cells. Nat Biotechnol 32, 554-561 (2014).
5. Chang, C.-W., Lai, Y.-S., Lamb, L. S. & Townes, T. M. Broad T-Cell Receptor Repertoire in T-Lymphocytes Derived from Human Induced Pluripotent Stem Cells. PLoS One 9, (2014).
6. Nishimura, T. et a/. Generation of Rejuvenated Antigen-Specific T Cells by Reprogramming to Pluripotency and Redifferentiation. Cell Stem Cell 12, 114-126 (2013).
7. Themeli, M. et al. Generation of tumor-targeted human T lymphocytes from induced pluripotent stem cells for cancer therapy. Nat Biotechnol 31, 928-933 (2013).
8. Vizcardo, R. et al. Regeneration of Human Tumor Antigen-Specific T Cells from iPSCs Derived from Mature CD8+ T Cells. Cell Stem Cell 12, 31-36 (2013).
9. Montel-Hagen, A. et al. Organoid-induced differentiation of conventional T cells from human pluripotent stem cells. Cell Stem Cell 24, 376-389. e8 (2019).
10. Guo, R. et al. Guiding T lymphopoiesis from pluripotent stem cells by defined transcription factors. Cell Research 30, 21-33 (2020).
11. Nagano, S. et al. High Frequency Production of T Cell-Derived iPSC Clones Capable of Generating Potent Cytotoxic T Cells. Molecular Therapy—Methods & Clinical Development 16, 126-135 (2020).
12. Iriguchi, S. et al. A clinically applicable and scalable method to regenerate T-cells from iPSCs for off-the-shelf T-cell immunotherapy. Nature Communications 12, 430 (2021).

1. A method for preparing a B cell population or progenitors thereof, the method comprising:
enriching for CD34+ cells from a differentiated pluripotent stem cell (PSC) population to prepare a CD34+-enriched population;
inducing endothelial-to-hematopoietic transition of the CD34+-enriched cell population for at least two days, but no more than 12 days, to prepare a population comprising hematopoietic stems cells (HSCs) and/or hematopoietic stem progenitor cells (HSPCs); and
differentiating the population comprising HSCs and/or HSPCs to a progenitor B cell population or a B cell population.

2. The method of claim 1, wherein the PSC population is a human iPSC population derived from lymphocytes, cord blood cells, peripheral blood mononuclear cells, CD34+ cells, or human primary tissues.

3. The method of claim 2, wherein the iPSC population is derived from CD34+ cells isolated from peripheral blood.

4. The method of claim 2 or 3, wherein the iPSCs are homozygous for one or more HLA Class I and/or Class II genes.

5. The method of claim 4, wherein the iPSCs are homozygous for HLA-DRB1.

6. The method of claim 4, wherein the iPSCs are homozygous for both HLA-B and HLA-C.

7. The method of any one of claims 2 to 4, wherein the iPSCs are gene-edited to delete one or more HLA Class I genes, delete one or more Class II genes, and/or delete one or more genes governing HLA or MHC expression or presentation capacity.

8. The method of claim 7, wherein the iPSCs comprise a deletion of HLA-A.

9. The method of claim 7 or 8, wherein the iPSCs comprise a deletion of HLA-DPB1 and/or HLA-DQB1.

10. The method of any one of claims 2 to 9, wherein the iPSCs are gene edited to be HLA-A^{neg}, homozygous for both HLA-B and HLA-C, HLA-DPB1^{neg}, and HLA-DQB1^{neg}, and optionally further homozygous for HLA-DRB1.

11. The method of claim 7, wherein the one or more genes governing HLA or MHC expression or presentation capacity is β2-microglobulin and/or CIITA.

12. The method of any one of claims 1 to 11, wherein CD34+-enrichment and endothelial-to-hematopoietic transition is induced at Day 8 to Day 15 of iPSC differentiation.

13. The method of any one of claims 1 to 12, wherein the CD34+-enriched population is cultured in medium containing Y-27632, TPO, IL-3, SCF, IL-6, IL-11, IGF-1, VEGF, bFGF, BMP4, and FLT3.

14. The method of claim 12 or 13, wherein the endothelial-to-hematopoietic transition generates an HSC population comprising one or more of long-term hematopoietic stem cells (LT-HSCs), short-term hematopoietic stem cells, and hematopoietic stem progenitor cells.

15. The method of any one of claims 12 to 14, wherein CD34+ cells are harvested from culture undergoing endothelial-to-hematopoietic transition, including harvesting of CD34+ floater and/or adherent cells.

16. The method of any one of claims 1 to 15, wherein the HSC population comprises long-term hematopoietic stem cells (LT-HSCs)

17. The method of any one of claims 1 to 16, where the induction of endothelial-to-hematopoietic transition comprises increasing the expression or activity of dnmt3b.

18. The method of claim 17, wherein the induction of endothelial-to-hematopoietic transition comprises applying cyclic stretch to the CD34-enriched cells.

19. The method of claim 18, wherein the cyclic stretch is 2D, 3D, or 4D cyclic stretch.

20. The method of any one of claim 1 to 16, wherein the induction of endothelial-to-hematopoietic transition comprises Piezol activation.

21. The method of claim 20, wherein the Piezol activation is by contacting the CD34+ enriched cells or fraction thereof with one or more Piezol agonists, which are optionally selected from Yoda1, ssRNA40, Jedi1, Jedi2, or analogues or derivatives thereof.

22. The method of any one of claims 1 to 16, wherein the induction of endothelial-to-hematopoietic transition comprises Trpv4 activation.

23. The method of claim 22, wherein the Trpv4 activation is by contacting the CD34+ enriched cells with one or more Trpv4 agonists, which are optionally selected from GSK1016790A, 4alpha-PDD, or analogues or derivatives thereof.

24. The method of any one of claims 1 to 23, wherein the B cell lineage is selected from multipotent progenitor cells (MPPs), common lymphoid precursor (CLP), common lymphoid 2 progenitor (LCA-2), early pro-B cells, late pro B cells, pre B cell, immature B cells.

25. The method of any one of claims 1 to 24, wherein the B cell lineage can differentiate into one or more lineages with phenotypes consistent with transitional B cells, regulatory B cells, marginal zone B cells, follicular B cells, activated B cells, memory B cells or plasma B cells or a combination thereof.

26. The method of claims 24 or 25, wherein the B cell lineage expresses a chimeric antigen receptor (CAR).

27. The method of claim 26, wherein the CAR-modified B cell lineage of immune cells is selected from one or more of a CAR-transitional B cells, CAR-regulatory B cells, CAR-marginal zone B cells, or a CAR-follicular B cells, or CAR-activated B cells, CAR-memory B cells or CAR-plasma B cells.

28. A B cell lineage cell population, or pharmaceutically-acceptance composition thereof, produced by the method of any one of claims 1 to 27.

29. A B cell population, or pharmaceutically-acceptance composition thereof, wherein the B cell population is HLA-A^{neg}, homozygous for both HLA-B and HLA-C, HLA-DPB1^{neg}, and HLA-DQB1^{neg}, and optionally further homozygous for HLA-DRB1.

30. A method for cell therapy, comprising administering the B cell lineage cell population or pharmaceutically acceptable composition thereof of claim 28 or claim 29, to a human subject in need thereof.

31. The method of claim 30, wherein the human subject has a condition comprising one or more of lymphopenia, a cancer, an immune deficiency, an autoimmune disease, viral infection, a skeletal dysplasia, and a bone marrow failure syndrome.

32. The method of claim 31, wherein the subject has cancer, which is optionally a solid tumor.
